(19) United States
(12) Patent Application Publication
(10) Pub. No.: US 2023/0417738 A1
(43) Pub. Date: Dec. 28, 2023

(54) Mass Spectrometric Determination of Cell Toxicity

(71) Applicant: Bruker Daltonics GmbH & Co. KG, Bremen (DE)

(72) Inventors: Evgeny IDELEVICH, Greifswald (DE); Karsten BECKER, Hanshagen (DE)

(21) Appl. No.: 18/212,715

(22) Filed: Jun. 22, 2023

(30) Foreign Application Priority Data
Jun. 22, 2022 (DE) 102022115561-7

(51) Int. Cl. G01N 33/50

(52) U.S. Cl. CPC G01N 33/5014

(57) ABSTRACT

The invention relates to a method for determining the cytotoxic effect of an analytical sample on animal cells, including human cells. The method comprises (a) a sample provision step in which a mass spectrometric sample comprising animal cells, nutrient medium, and the analytical sample potentially having a cytotoxic factor is prepared on at least one sample spot of a mass spectrometric sample support; (b) a cultivation step in which the mass spectrometric sample is incubated on the sample spot of the mass spectrometric sample support in a cultivation device; (c) a liquid removal step in which residual liquid of the mass spectrometric sample is removed from the sample spot; (d) a measuring step in which spatially resolved mass spectra are recorded at a plurality of measuring positions in at least a partial area of the sample spot by means of a spatial resolution mass spectrometer, wherein, when a matrix-based spatial resolution mass spectrometer is used, prior to the recording of the spatially resolved mass spectra in the measuring step, a preparation of the sample spot is carried out in a preceding sample preparation step by the spatially dispersed application of a matrix to at least the one partial area of the sample spot; (e) a first evaluation step in which each spatially resolved mass spectrum is analyzed for the presence of a cell-specific mass spectrometric signature for the animal cells and a cell presence value is assigned to each measuring position; (f) a second evaluation step in which a degree of coverage by animal cells is determined for at least the partial area of the sample spot from the cell presence values of the measuring positions; (g) and/or a third evaluation step in which a proliferation capability is derived from the determined degree of coverage; (h) a fourth evaluation step in which the cytotoxic effect of the analytical sample on the animal cells is derived indirectly or directly from the determined degree of coverage of the second evaluation step, wherein for this purpose the cytotoxic effect of the analytical sample on the animal cells is derived from the result of at least one of the two steps: second evaluation step and/or third evaluation step.

### CROSS REFERENCE TO RELATED APPLICATIONS

The present application claims priority under 35 U.S.C. § 119 to German Patent Application Serial No. 102022115561-7 filed on Jun. 22, 2022, which is hereby incorporated by reference in its entirety.

### DESCRIPTION

The present invention relates to a mass spectrometric method for determining the cytotoxic effect of cytotoxic factors on animal cells.

Two main tasks of routine microbiology laboratories are the identification of microbial pathogens and antimicrobial susceptibility testing (i.e. resistance testing) of bacteria and fungi.

In recent years, various mass spectrometric methods have found their way into research laboratories and routine microbiological laboratories. Especially in microbiology laboratories, a special mass spectrometry (MS) method, “matrix-assisted laser desorption ionization-time of flight” (MALDI-ToF) mass spectrometry, has become established in recent years and most microbiology laboratories have been equipped with appropriate MALDI-ToF MS instruments over the last decade. However, the routine application of this method has so far focused exclusively on the pathogen identification of bacteria and fungi. In addition, there are the first applications for determining the resistance of bacteria and fungi. So far, most of these are still in the development stage. For example, WO2018/099500A1, Idelevich et al. [E. A. Idelevich, K. Sparbier, M. Kostrzewa, K. Becker, Rapid detection of antibiotic resistance by MALDI-ToF mass spectrometry using a novel direct-on-target microdroplet growth assay, Clinical Microbiology and Infection, Volume 24, Issue 7, 2018.] describes the cultivation of microbial samples on MS sample supports and the determination of the resistance of microorganisms to microbial substances.

However, besides the identification of bacteria and fungi and the determination of the resistance of bacteria and fungi to certain antimicrobial active substances, there are other aspects to be investigated in the laboratories where MS is not applied. In addition to molecular biological detection methods, such as PCR, these are based on phenotypic detection methods, in which the effect of cytotoxic factors is detected via the growth of animal cells, e.g., continuous cell lines, usually optically (i.e., either by detecting a color reaction or by microscopically determining the vitality and morphology of the cells).

An important aspect to be investigated is, for example, the virulence of a pathogen. Virulence is the degree of pathogenicity of a particular isolate of a microbial species, where pathogenicity means the basic ability of pathogens to cause disease in a macroorganism (host, for example humans). A distinction can be made here between targeted (hypothesis-based) detection of specific relevant virulence phenotypes and universal (non-hypothesis-based) phenotypic determination of virulence.

Another important aspect is a determination of the sensitivity of viruses to antiviral agents and a determination of the cell cytotoxicity of an antiviral agent. Similar to the administration of antibiotics for bacterial infections, the administration of antivirals is usually started empirically and adjusted as needed according to the results of antiviral resistance testing. This means that in case of resistance to an antiviral agent (for example, a virostatic agent), a switch is made to another antiviral agent. Not infrequently, the absence of an improvement in the condition of the treated organism is the first indication of a suspicion of the pathogen's resistance and thus gives cause for antiviral susceptibility testing. Phenotypic tests can also help here, provided such are available.

In addition to the detection of the virulence of bacteria, the determination of the sensitivity of viruses to antiviral agents, and the determination of the cell toxicity of antibacterial, antifungal, or antiviral agents, the detection of the sensitivity of tumor cells to chemotherapeutic agents is also an area of application of phenotypic tests.

### Various Methods are Available for the Detection of Cytotoxicity or Virulence.

WO2018/099500 discloses the detection of growth of microorganisms using a simple MALDI-ToF. This is possible because the microorganisms have a high growth and cell division rate. However, it has been shown that the above-mentioned methods, which were developed for microorganisms, are poorly suited for the detection of growth of animal cells, since, among other things, the growth and cell division of animal cells is usually significantly slower than the growth and cell division of microorganisms and the cell numbers are generally much lower.

Usually, animal cell tests for determining virulence and cytotoxicity of substances are based on one of the following methods:
molecular biological methods
a determination of the viability (viability as the number of living cells in a cell population) of the animal cells through
   analysis of metabolic activity by staining (phenotypic method) or MS
   detection of specific pharmacodynamic biomarkers using MS
   cell counting of animal cells or determination of the confluence or other morphological parameters of animal cells by microscopic/imaging methods (phenotypic method)

For example, in many cases, the detection of microbial virulence is still done phenotypically. Genotypic detection by molecular biological methods (e.g. polymerase chain reaction, PCR) is only possible in exceptional cases, where the virulence genes are known (e.g., Panton-Valentine leucocidin and Shiga toxin). However, most virulence characteristics are not encoded by a specific gene, but by a plurality of mechanisms, some of which have not yet been completely decoded by molecular biology (genotyping). In addition, the regulation thereof in the pathogen is highly complex, so that the so-called genotype (totality of the coded genetic material) does not necessarily correspond to the phenotype (distinct characteristics). Therefore, a comprehensive virulence determination is only possible by means of phenotypic detection methods, which, however, are both complex and cost-intensive and require a relatively long time until the result is obtained. Consequently, virulence determination is currently reserved almost exclusively for research laboratories, as these can meet the requirements necessary for universal virulence determination, such as costly instruments and complex methods, but routine laboratories usually cannot.

The metabolic activity can be detected, for example, by means of the MTT cytotoxicity test [ISO 10993-5:2009 (E)]. In the MTT test, the yellow substance MTT (3-(4,5-dimethylthiazol-2-yl)-2,5-diphenyltetrazolium bromide) is metabolically reduced by the viable cells to the blue-violet substance formazan. The number of viable cells correlates with the photometrically measured color intensity. Semi-high-throughput UV spectrophotometers are usually used for optical detection of the staining. However, the use of these tests is significantly hampered by at least two problems. One is the long duration currently needed to perform a cytotoxicity test. For the MTT test mentioned above, the time to result is about three days. Secondly, the performance of cytotoxicity tests is technically extraordinarily complex and requires the availability of special equipment, so that these tests must be reserved for highly specialized laboratories.

In addition to phenotypic methods, indirect MS methods are used to specifically detect the effect of active substances by detecting specific individual pharmacological biomarkers in eukaryotic cells.

The detection of individual pharmacodynamic biomarkers using MALDI-ToF has been described by Unger M. et al. [Direct Automated MALDI Mass Spectrometry Analysis of Cellular Transporter Function: Inhibition of OATP2B1 Uptake by 294 Drugs, Anal. Chem. 2020.] Eukaryotic cells on MALDI supports were described by Bergquist, J. [Cells on the target matrix-assisted laser-desorption/ionization time-of-flight mass-spectrometric analysis of mammalian cells grown on the target. Chromatographia 49, p. 41-48 (1999).] The samples were analyzed with a view to discovering new peptides and proteins.

In addition, for the detection of individual pharmacological biomarkers to study the effect of the drug, methods with high spatial resolution MALDI-MSI are also used to visualize the effect in individual cells [Munteanu B. et al., Label-Free in Situ Monitoring of Histone Deacetylase Drug Target Engagement by Matrix-Assisted Laser Desorption Ionization-Mass Spectrometry Biotyping and Imaging, Analytical Chemistry, 2014. Ramallo, C. et al., Fast Nanoliter-Scale Cell Assays Using Droplet Microarray—Mass Spectrometry Imaging, Adv. Biology; 2021.] For this, however, not only the specific pharmacological biomarker (e.g., metabolite change) but also the direct relation to the active substance must be known. Another disadvantage is that one pharmacological biomarker cannot be used universally for all classes of active substances. In addition, it is disadvantageous that the detection of biomarkers usually does not allow any or any reliable statement about the viability of the cell or the cytotoxicity of a substance. Furthermore, cytotoxic factors in particular, such as bacteria, fungi, or viruses, are difficult to detect using biomarkers from animal cells. In addition, high-spatial-resolution MALDI MSI systems are very expensive and available in few routine laboratories.

Therefore, cell counting as well as the determination of the confluence of the cells (or the degree of coverage) is used as an alternative. These are performed using live cell imaging devices, which use microscopy modules, image and data acquisition, and powerful image and data analysis to reliably detect the growth and division ability of the cells [Carlsen J. et al., Optimized High-Contrast Brightfield Microscopy Application for Noninvasive Proliferation Assays of Human Cell Cultures, Assay and Drug Development Technologies, July 2020.215-225]. The disadvantage here is that the required equipment is very cost-intensive, is available in only a few routine laboratories, and the staff must be specifically trained to operate the equipment. The use of simple microscopes would be cheaper in terms of equipment purchase, but microscopic assessment is time-consuming and subjective.

The objective of the invention is to overcome the described disadvantages of the prior art and, in particular, to provide an alternative method for determining the presence of cytotoxic factors in an analytical sample of which it is not known whether a (or multiple) cytotoxic factor is present, which method is reliable, simple, fast, and inexpensive. A further object of the invention to determine the effect of potentially cytotoxic factors on animal cells and the toxicity thereof reliably, simply, quickly, and inexpensively. It is further an object of the invention to provide a system for carrying out such a method.

The objective is achieved by means of a method having the features of independent claim 1, a method having the features of independent claim 2, a method having the features of independent claim 3, and a system having the features of independent claim 21. Advantageous embodiments can be found in the dependent claims.

By providing a method for determining the cytotoxic effect of an analytical sample on animal cells, comprising the following steps:
a sample provision step A in which a mass spectrometric sample comprising animal cells, culture medium, and the analytical sample potentially having a cytotoxic factor is provided on at least one sample spot of a mass spectrometric sample support;
a cultivation step in which the mass spectrometric sample is incubated on the sample spot of the mass spectrometric sample support in a cultivation device;
a liquid removal step, in which residual liquid of the mass spectrometric sample is removed from the sample spot;
a measuring step A, in which spatially resolved mass spectra are recorded at a plurality of measuring positions in at least a partial area of the sample spot by means of a spatial resolution mass spectrometer, wherein, when using a matrix-based spatial resolution mass spectrometer, prior to the recording of the spatially resolved mass spectra in the measuring step A, in a preceding sample preparation step A a preparation of the sample spot is carried out by a true-to-position application of a matrix to at least the one partial area of the sample spot;
a first evaluation step A in which each spatially resolved mass spectrum is analyzed for the presence of a cell-specific mass spectrometric signature for the animal cells and a cell presence value is assigned to each measuring position;
a second evaluation step A, in which a degree of coverage by animal cells is determined for at least the partial area of the sample spot from the cell presence values of the measuring positions;
and/or a third evaluation step A in which a proliferation capability is derived from the determined degree of coverage;
a fourth evaluation step A, in which the cytotoxic effect of the analytical sample on the animal cells is derived indirectly or directly from the determined degree of coverage of the second evaluation step A, wherein for this purpose the cytotoxic effect of the analytical sample on the animal cells is derived from the result of at least one of the two steps: second evaluation step A, third evaluation step A;
   the presence of cytotoxic factors in an analytical sample can be checked reliably, simply, quickly, and inexpensively. In addition, the effect of potentially cytotoxic factors on animal cells can be tested and the toxicity thereof can be determined reliably, simply, quickly, and inexpensively.

It has been shown that spatial resolution mass spectrometers can be used to determine the cytotoxic effect of analytical samples. Animal cells generally grow and divide much more slowly compared to microorganisms, resulting in only a slow increase in cell number at a sample spot, if any. Thus, cultivation of animal cells from mass spectrometric samples for non-spatial-resolution mass spectrometers, which generate a sum mass spectrum per sample spot, must be carried out for a correspondingly long time in order to obtain sufficient cell quantities over the entire sample spot. This prolongs the analysis time and makes such a procedure impractical for the application. Also, the sum-mass spectrum of a sample spot is often ambiguous, because mass spectrometric signals from animal cells that have been provided before cultivation (cell seed) make the detection of a cytotoxic effect on animal cells more difficult. On the other hand, it has been recognized that the areal expansion of animal cells on a sample spot, and thereby the proliferation capability thereof, and finally a cytotoxic effect can be determined in order to avoid the previously described disadvantages in determining the cytotoxic effect of analytical samples. Preferably, the areal extent of the animal cells on a sample spot, the proliferation ability, the migration ability, and/or the cytotoxic effect is determined by means of a spatial resolution mass spectrometer. For this purpose, the degree of coverage at a certain point in time of a sample spot and/or the proliferation capability derived therefrom is preferably determined. Particularly preferably, the extension of the animal cells on the sample spot is determined by determining the degree of coverage at a specific time. Preferably, the total area of the sample spot is known.

This means that cell growth and cell proliferation can also be detected quickly in animal cells. In particular, this can be used especially for adherent proliferating cells. Preferably, therefore, the animal cells are adherently proliferating (adherently growing) animal cells.

Detection by means of a spatial resolution mass spectrometer is also faster and simpler than the microscopic or imaging methods previously used for this purpose, and in certain cases it is also cheaper. For example, mass spectrometers already available in laboratories are often capable of performing the procedure through software modification. In addition, the process can be used universally.

In order to prevent an uneven sample distribution on the sample spot for statistical purposes and for reasons of measurement accuracy, measurements are usually already carried out at several measuring positions of a sample spot even for non-spatial-resolution MALDI-ToFs, but these are summed up to a sum parameter/MS spectrum during data processing or evaluation. The spatial information thus remains unused here. Thus, for many non-spatial-resolution MALDI ToFs, the device-related prerequisite is usually present. It has been recognized that an adjustment of the software is often sufficient to convert a non-spatial-resolution MALDI-ToF-MS into a spatial resolution MALDI-ToF-MS for the described method. Furthermore, it has been recognized that almost any cell-specific mass spectrometric signatures of the animal cell are suitable for the detection of the animal cells and thus the cytotoxic effect, since only the presence or absence of the cell have to be confirmed for several measuring positions in order to conclude a cytotoxic effect. Only the presence or absence of the cell must be detected. Specific pharmacological biomarkers, such as an increase in certain metabolites, are not needed. For example, changes in concentrations of biomarkers are not tracked in order to draw conclusions about the effect on the metabolic state of the cell. Thus, no knowledge of specific metabolic activities or specific biomarkers metabolically related to the cytotoxic factor, for example, is required. Information about the cytotoxic factor itself and the mode of action thereof on animal cells, that is, for example, on metabolism, is also not required. Also, knowledge about interactions in the case of a plurality of cytotoxic factors present is not necessary. The cytotoxic factor does not even have to be known. This makes the method universally applicable for cytotoxic factors.

One or more cell-specific mass spectrometric signatures are used to infer the presence of a cell at a measuring position. This is in turn used to determine the degree of coverage (claim 1 and claim 2) or the cell expansion distance (claim 3) to infer the proliferative capability of the cell, the migratory capability of the cell, and/or the cytotoxic effect of an analytical sample.

In a preferred application of the method, it is unknown at the outset whether an analytical sample comprises a cytotoxic factor having a cytotoxic effect. The presence of a cytotoxic effect on the animal cells is determined by means of the procedure. The detection of a cytotoxic effect on the animal cells by means of the method indicates the presence of at least one cytotoxic factor.

The presence of a virulent pathogen in the analytical sample can be directly deduced from the cytotoxic effect on the animal cells or the presence of a cytotoxic factor.

In a further preferred application of the method, it is determined whether a specific potentially cytotoxic factor is a cytotoxic factor having an actual cytotoxic effect on the animal cells. On the other hand, an effect of known cytotoxic factors on a certain type of animal cell can also be tested on further types of animal cells with this method. Furthermore, factors that potentially neutralize the cytotoxic factor can also be tested for the effect thereof on the cytotoxic factor. For example, the method according to claims 1, 2, and/or 3 and/or subclaims thereof and/or the system according to claim 21 may be used for virulence phenotype determination. Medicines or substances the effect of which is unknown can also be tested for the effect thereof. The method is particularly advantageous in the detection of complex, multi-causal virulences. Thus, the method is also suitable for detecting the cytotoxic effect in the presence of multiple cytotoxic factors only showing the cytotoxic effect thereof in interaction.

It has also been recognized that when using a matrix-based mass spectrometer, it is necessary to apply the matrix to the sample in a true-to-position manner so that the spatial resolution of the mass spectrometric signal in the area, i.e., the position of the animal cells on the sample spot, is not distorted and cell presence values for each measuring position and/or the degree of coverage can be reliably determined in this method. In the case of true-to-position application of the matrix, the matrix is applied in such a way that a two-dimensional mixing of the cell contents and cell components of the animal cells on the sample spot is (largely) avoided.

Preferably, the claimed processes comprise the respective process steps. Alternatively preferably, the claimed processes consist of the respective process steps. Preferably, the claimed processes are carried out in the order of the wording of the claim.

Spatially resolved mass spectra are generated by the spatial resolution mass spectrometer. Spatially resolved means preferably resolved in the plane. The measuring positions are targeted specifically for the measurement. Preferably, the location coordinates of the measuring positions are recorded. Preferably, the spatial coordinates of the measuring positions are assigned to each spatially resolved mass spectrum. The spatial resolution mass spectrometer preferably specifically measures several measuring positions of a sample spot and preferably assigns the spatial coordinates of the respective associated measuring position to each recorded mass spectrum. Alternatively, preferably, the spatial resolution mass spectrometer assigns spatial coordinates to each measuring position of a sample spot and preferably assigns a measuring position to each recorded mass spectrum. Thus, each spatially resolved mass spectrum generated can be assigned to a defined measuring position on the sample spot. Furthermore, the position coordinates of the measuring positions are preferably stored. However, in a particular alternative embodiment of the invention according to claims 1 and 2, it may be sufficient to assign the spatially resolved mass spectra to the measuring positions of a sample spot without recording the exact location coordinates of the measuring position. The decisive factor is that the measuring positions are measured by the spatial resolution mass spectrometer in a targeted manner, i.e., according to the specified measuring positions, and the individual mass spectra of the measuring positions are not calculated into a total/sum mass spectrum but are assigned to the respective measurement.

According to claim 3, the spatial coordinates of each measuring position are recorded and assigned to the spatially resolved mass spectra.

The spatial resolution mass spectrometer may be a matrix-based spatial resolution mass spectrometer or a non-matrix-based spatial resolution mass spectrometer. The matrix-based spatial resolution mass spectrometers are mass spectrometers with matrix-based ionization processes. This means that said spectrometers use a matrix, usually applied in liquid form, to ionize the molecules of the mass spectrometric sample. These include, for example, MALDI-ToF mass spectrometers. The non-matrix based spatial resolution mass spectrometers are mass spectrometers with non-matrix based ionization methods. This means that said spectrometers do not use a matrix to ionize the molecules of the mass spectrometric sample. These include DESI mass spectrometers, for example. Preferably, the spatial resolution mass spectrometer is a matrix-based spatial resolution mass spectrometer. Preferably, the spatial resolution mass spectrometer is a matrix-based spatially resolved MALDI-ToF MS. To this end, the MALDI-ToF MS is configured to be able to record spatially resolved mass spectra. Further preferably, the spatial resolution mass spectrometer is a MALDI-ToF MS with imaging function, which allows the acquisition of spatially resolved mass spectra with a high areal density and thus precise measurements. Said spectrometers are easy to handle and fast.

Furthermore, non-spatial-resolution MALDI-ToF MS can be converted to spatial-resolution MALDI-ToF MS by simple software conversion. Simple non-spatial-resolution MALDI-ToF MS have the advantage to being relatively inexpensive to purchase, easy to operate and are already established in many laboratories, e.g. for the identification of microorganisms.

The adhesion and proliferation of the animal cells in the process can take place in one process step during the cultivation step. The adhesion and proliferation of the animal cells can also take place in two steps, in that the animal cells are provided already adhered in sample provision step A, B and only the proliferation of the adhered animal cells takes place in the cultivation step. In a preferred embodiment, when the mass spectrometric sample is provided in sample provision step A, B, components of the mass spectrometric sample, such as the animal cells, the culture medium and/or the cytotoxic factor is already present at the sample spot. This can be done, for example, in dried, cooled and/or frozen form. Thus, a mass spectrometric sample support can be made commercially available to customers already with proliferation-capable animal cells. In a preferred embodiment, when the mass spectrometric sample is prepared in sample provision step A, B, components of the mass spectrometric sample, such as the animal cells, the culture medium and/or the cytotoxic factor is already present at the sample spot. At least one component of the mass spectrometric sample spot is provided in this preferred embodiment dissolved or suspended in a liquid, preferably buffer, nutrient medium or water. In a particularly preferred embodiment, the animal cells and the culture medium are already present and the analytical sample or the cytotoxic factor is applied. For example, the animal cells with the culture medium can be freeze-dried. In addition, the sample spot may be provided with a water-soluble adhesive to keep the dried or freeze-dried animal cells and culture medium localized on the sample spot. In another preferred embodiment, all components of the mass spectrometric sample, comprising animal cells, the culture medium and the cytotoxic factor, are applied to the sample spot. Preferably, the animal cells are suspended in a liquid culture medium.

The animal cells are viable and, proliferative and/or migratory at least prior to application of the analytical sample or cytotoxic factor.

The culture medium includes all the components that the animal cells need for proliferation and/or migration. The culture medium can be synthetic or complex. Furthermore, the culture medium can also contain components that additionally enable the cultivation of bacteria or fungi as cytotoxic factors.

The cultivation of the mass spectrometric sample on the sample spot of the mass spectrometric sample support in the cultivation device in the cultivation step serves to create optimal cultivation conditions to enable proliferation of the animal cells. Preferably, the cultivation of the mass spectrometric samples including the reference samples is completed simultaneously. This makes it easy to determine the degree of coverage and/or the cell expansion distance at a specific point in time. This makes it easy to determine the proliferation capability and/or migration capability. The duration of cultivation and the cultivation conditions are adapted to the animal cells. Preferably, the cultivation time is selected in such a way that, in the case of a reference sample without cytotoxic factor, the partial area or the entire sample spot is largely completely covered with animal cells at the end of the cultivation. For this purpose, the cultivation device preferably provides optimal growth conditions for the animal cells. These include humidity, temperature, carbon dioxide content and oxygen content. Thus, the cultivation device controls the environmental conditions such as humidity, temperature, CO₂ content in the cultivation device, O₂ content in the cultivation device, and/or other environmental conditions. CO₂ incubators, in particular can serve as cultivation equipment. Alternatively, the cultivation device can be set to conditions similar to those of the human or animal body from which the original sample was obtained. Preferably, the air in the cultivation device is saturated with water in such a way that the liquid of the mass spectrometric sample, in particular the culture medium, does not substantially evaporate.

After the animal cells have had the conditions and time to grow, divide and/or migrate, the residual fluid is removed. The residual liquid is a mixture of the liquid components of the mass spectrometric sample. For example, the animal cells were suspended and/or the cytotoxic factor was dissolved in them. The liquid components of the mass spectrometric sample may include the culture medium, buffers, water, blood, urine, secretions and other liquid components. The residual liquid is preferably removed by contact with an absorbent material and/or by drying. Drying can be done e.g., by simple air drying, specific ventilation and/or by thermal drying. By removing the residual liquid, especially the culture medium, the cell-specific mass spectrometric signals are easier to detect. In addition, a largely dry surface is advantageous for the localized application of a matrix. It is crucial that sufficient residual liquid is removed so that, in the case of non-matrix-based spatial resolution mass spectrometers, a measurement is not negatively influenced by liquid residues and, in the case of matrix-based spatial resolution mass spectrometers, there is no longer a liquid film on the animal cells, which would make a true-to-position application of the matrix impossible. Preferably, the residual liquid is sucked up when using adherent growing animal cells. This largely prevents animal cells in suspension from depositing on the sample spot and falsifying the result. This increases the accuracy of the process. Also, interfering non-cell-specific mass spectrometric signals from the culture medium and the remaining components of the mass spectrometric sample are reduced. Alternatively, the residual liquid will be removed by drying. This can be advantageous when using suspension-grown animal cells, among other things, so that the suspended cells can sediment onto the sample spot and adsorb to the surface. In principle, drying for liquid removal is also possible when adherent growing cells are applied in suspended form, if the initial cell number and the cultivation time are adjusted according to the remaining non-adherent animal cells on the sample spot. Especially when using matrix-based spatial resolution mass spectrometers, drying is preferably conducted after suction in order to reliably remove the residual liquid. This increases the resolution when the matrix is subsequently applied spatially.

When using a matrix-based spatial resolution mass spectrometer, a sample preparation step A is preceded by the measurement step A. This step is missing in non-matrix-based spatial resolution mass spectrometers. In sample preparation step A, the sample spot is prepared by applying a matrix to at least a partial area of the sample spot. Thereby, a mixing of the analytes of one measuring position of the sample spot with analytes of other measuring positions of the sample spot is largely avoided. All analytes remain largely in place.

In the case of true-to-position application, the matrix is not applied in the form of one or a few large drops to the entire sample spot. Instead, the application is conducted in numerous smaller portions, preferably either by droplets or by sublimation, distributed over the sample spot. The application of the matrix is preferably carried out by applying a large number of matrix droplets per sample spot. In this case, the plurality of matrix droplets are preferably applied over the sample spot or the partial area of the sample spot in a distributed manner. The application of the matrix droplets is targeted or untargeted depending on the application method. As a result, at the end of the application process, the entire partial area of the sample spot or the entire sample spot is preferably covered with matrix droplets, so that all animal cells of the partial area or the entire sample spot are accessible for mass spectrometric determination. It is crucial for the true-to-position application of the matrix that the animal cells are not covered by a droplet spanning the sample spot.

The matrix usually causes, among other things, a breakdown of the cells. The true-to-position application of the matrix ensures that the cell content and the fragments of each animal cell do not spread over large parts of the sample spot and cover measuring positions that do not contain cells. This prevents falsification of the measurement. Due to a diffusion-driven broad distribution of the cell contents over the entire sample spot in the case of application of a large matrix droplet to the entire sample spot, an exact spatial resolution of the positions of the cells would no longer be possible. Preferably, the diameter of the matrix droplets is significantly smaller than the diameter of the animal cells. This supports that the cell content remains largely within the (original) cell boundary. Matrix application is not required for non-matrix-based MS, i.e. MS with ionization methods such as DESI or SIMS. By recording spatially resolved mass spectra at a large number of measuring positions in at least a partial area of the sample spot by means of a spatial resolution mass spectrometer in measuring step A, each measured measuring position on the sample spot is assigned a dedicated mass spectrum for the following evaluation steps. A partial area of the sample spot or the entire sample spot can have measuring positions. Preferably, the sample spot or the partial area of the sample spot is not measured over the entire surface, but is measured randomly in the form of a grid—i.e. scanned. The number of measuring positions and/or the grid of measuring positions depends on the spatial resolution mass spectrometer as well as on the desired precision and speed of the method. Preferably, a partial area of the sample spot is systematically scanned. This speeds up the procedure. Alternatively, the entire sample spot can be systematically scanned. This increases the reliability of the process.

By analyzing each spatially resolved mass spectrum for the presence of a cell-specific mass spectrometric signature for the animal cells in the first evaluation step A, the data basis is created for the subsequent determination of the degree of coverage or the cell expansion distance. Each spatially resolved mass spectrum is preferably analyzed on its own. It is crucial that the mass spectra remain spatially resolved. Several mass spectra for the same measuring position can be offset. In particular, the calculation can include an averaging. However, mass spectra from different measuring positions are not combined.

Preferably, the cell-specific mass spectrometric signature consists largely or entirely of a selection of mass spectrometric signals in the recorded spatially resolved mass spectrum which can be unambiguously assigned to the animal cell. Further preferably, these signals have a significantly higher intensity than the background noise and/or than a plurality of the non-cell-specific mass spectrometric signals. The cell-specific mass spectrometric signals of the spatially resolved mass spectra of the animal cells may differ in part depending on the location of the measuring position on the animal cell or on the disrupted animal cell, but always indicate the presence of the animal cell. Preferably, cell-specific mass spectrometric signatures exhibit high reproducibility with changing analytical samples. Further preferably, cell-specific mass spectrometric signatures also exhibit high reproducibility across a variety of different animal cells. This increases the reliability of the process. The cell-specific mass spectrometric signals and/or cell-specific mass spectrometric signatures may also depend on the species/type of animal cell used. Said signals can be specific to certain tissue types, cell culture lines, organisms or further classification groups of cells.

Selected cell-specific mass spectrometric signatures of the entire spatially resolved mass spectrum can be combined into one or more cell-specific mass spectrometric signatures. The cell-specific mass spectrometric signals and cell-specific mass spectrometric signatures are preferably already available as reference values in a database of a data processing unit and can be used in the analysis.

Preferably, the same cell-specific mass spectrometric signature is used for all measuring positions of a sample spot. In an alternative embodiment, the cell-specific mass spectrometric signatures of individual measuring positions or sample spots may also differ from each other. Multiple cell-specific mass spectrometric signatures can also be used, having a diverse selection of cell-specific mass spectrometric signals. The decisive factor in the selection of the cell-specific mass spectrometric signature or mass spectrometric signatures is that it can be determined unambiguously for each measuring position whether an animal cell is present or not.

In a special case, the cell-specific mass spectrometric signature may consist of only one cell-specific mass spectrometric signal. This simplifies the analysis of the spatially resolved mass spectra, but can reduce the robustness of the method. In particular, the robustness of the method may be reduced when using biological source samples, such as stool samples, due to superposition of the cell-specific mass spectrometric signal with signals from components of the source sample. Preferably, therefore, the cell-specific mass spectrometric signature comprises a plurality of cell-specific mass spectrometric signals and/or multiple cell-specific mass spectrometric signatures are used. Especially for samples with a high proportion of non-cell-specific mass spectrometric signals with a similar m/Z ratio as the cell-specific mass spectrometric signals, multiple cell-specific mass spectrometric signals are beneficial for the cell-specific mass spectrometric signature. Preferably, the most prominent mass spectrometric signals or the most prominent mass spectrometric signal of the cell-specific mass spectrometric signals are used for the cell-specific mass spectrometric signature. Preferably, the one or more cell-specific mass spectrometric signals have a high intensity relative to the background noise and/or are sufficiently spaced from non-cell-specific signals. High intensity relative to background noise means that the intensity of a mass spectrometric signal is at least twice as high as the average background noise. Preferably, the mass spectrometric signal is at least five times higher than the average background noise. Further preferably, the mass spectrometric signal is at least ten times higher than the average background noise. Particularly preferably, the mass spectrometric signal is at least times higher than the average background noise. In addition, the cell-specific mass spectrometric signals of the cell-specific mass spectrometric signature are preferably sufficiently spaced from non-cell-specific mass spectrometric signals. Preferably, the cell-specific mass spectrometric signature consists at least in part or completely of adjacent cell-specific mass spectrometric signals. However, it may also include non-directly adjacent cell-specific mass spectrometric signals.

Depending on which part of the cell the spatially resolved mass spectrum has been recorded, the mass spectra obtained may differ. In a further preferred embodiment, different cell-specific mass spectrometric signatures can therefore be used to determine a cell presence.

In another special case, the cell-specific mass spectrometric signature may consist of all cell-specific mass spectrometric signals in the recorded spatially resolved mass spectrum. In this special case, however, weighting is preferably used in the analysis step, since the presence of individual cell-specific mass spectrometric signals can be masked by non-cell-specific mass spectrometric signals, e.g. of the culture medium. Furthermore, the cell-specific signal or signature is preferably not or largely not generated by other components of the mass spectrometric sample, such as the culture medium or the cytotoxic factor. Thus, the cell-specific mass spectrometric signals of the cell-specific mass spectrometric signature are essentially absent from the mass spectra of the culture medium, the analytical sample or the other components in the mass spectrometric sample. This allows a simple and unambiguous statement as to whether animal cells were present at the measuring position or not.

Components of the mass spectrometric sample are, for example, the culture medium, the animal cells, the analytical sample, and other components that are required for the cultivation or the measurement. Furthermore, components of the mass spectrometric sample also include parts of the components of the mass spectrometric sample. This can include buffers, water, solids, and other parts that make up the components of the mass spectrometric sample.

Furthermore, the animal cells have components of the animal cells of which the animal cells are composed or comprised. The components of the animal cells may include, for example, proteins, peptides, nucleic acids, lipids, saccharides, ribosomes, enzymes, and/or building blocks, fragments and/or metabolic products thereof, and/or the cytoplasm. Preferably, secreted substances of the cell do not count as components of the cell. Further preferably, the amount, presence or production in the living animal cell of the component of the animal cell which produces the cell-specific signal is not influenced by components of the mass spectrometric sample, in particular the cytotoxic factor.

Alternatively, the components of the mass spectrometric sample can also be measured individually. The cell-specific mass spectrometric signals can be determined by comparing the mass spectra of animal cells with the mass spectra of culture medium, cytotoxic factors and other components of the sample. For example, the cell-specific mass spectrometric signature is determined on two reference samples. In this case, the first reference sample has no animal cells, but includes the culture medium and the other components of the mass spectrometric sample. In this case, the second reference sample has all components of the mass spectrometric sample including the animal cells, but no cytotoxic factor or analytical sample. The comparison is carried out according to the mathematical procedures known to the experienced specialist in the field of mass spectrometric evaluation procedures.

Based on the analysis of each spatially resolved mass spectrum with regard to the presence of a cell-specific mass spectrometric signature for the animal cells in the first evaluation step A, B, it is determined whether or not animal cells or disrupted animal cells were present at the measured measuring positions during the measurement step A, B. The analysis of the cell-specific mass spectrometric signature for the animal cells in the first evaluation step A, B is based on the analysis of the cell-specific mass spectrometric signature. The result is assigned to the cell presence value. This is done analogously for mass spectra in the first evaluation step C and measurement step C for claim 2. Preferably, the cell presence value has two values: “Cell present” or “Cell not present”. A cell presence value is preferably assigned to each measured measuring position. From this, the degree of coverage or the cell expansion distance is determined. The evaluation of mass spectra, for example the analysis of the presence of certain peaks or intensities with subsequent evaluation of whether a cell is present or not, is also covered by the feature “Assignment of a cell presence value”. Preferably, the cell presence value does not have to be explicitly shown. Preferably, the determination that an animal cell is or is not present at a measuring position is sufficient to fulfil this characteristic.

By determining the degree of coverage by animal cells for at least the partial area of the sample spot from the cell presence values of the measuring positions in the second evaluation step A, C, the extent of the animal cells on the sample spot can be easily determined. Preferably, the degree of coverage is determined for the entire sample spot. Preferably, the total area of the sample spot is known.

The degree of coverage can be determined directly in the case of scanning the entire sample spot or extrapolated for the sample spot in the case of scanning a partial area. In the case of a determination of the degree of coverage for a partial area of the sample spot, the size of the partial area is preferably known.

The degree of coverage indicates what proportion of the area of the sample spot or part of the sample spot is or was covered with animal cells (in the case of disrupted cells). In a preferred embodiment, the degree of coverage is a degree of confluence. Here, the degree of coverage is determined by adherent-growing animal cells. In the simplest way, the degree of coverage can be determined by setting the number of measuring positions with a positive cell presence value in relation to the number of measuring positions with a negative cell presence value. Alternatively, the degree of coverage can also be determined by setting the cell presence values in relation to the partial area.

In another alternative preferred embodiment, the method comprises an additional third evaluation step A, in which a proliferation ability of the animal cells is derived from the determined degree of coverage. The cytotoxic effect of the cytotoxic factor in the fourth (preferably last) evaluation step is then preferably derived from the determined proliferation capability of the animal cells. By introducing this evaluation step, the proliferation capability can be determined in a simple way. Preferably, the proliferation capability is graded in degrees. This involves comparing the degree of coverage of the mass spectrometric sample with a degree of coverage of a reference sample or an existing reference value. The available reference values are preferably available in a database. Preferably, the reference sample comprises the animal cells and the culture medium. The proliferation level has at least 2 values. At the first value, the cells are not capable of proliferation; i.e., the cytotoxic factor has a cytotoxic effect on the animal cells. At the second value, the cells are fully proliferative; i.e. the proliferative capability of the animal cells is unaffected by the cytotoxic factor. One or more gradations between the two values, indicating a graded limited proliferation capability, may also be provided. Preferably, the proliferation capability is expressed as a percentage, where 100% corresponds to unhindered proliferation.

The cytotoxic effect of the analytical sample on the animal cells is derived indirectly or directly from the determined degree of coverage of the second evaluation step A, C in a fourth (preferably last) evaluation step. For this purpose, the cytotoxic effect of the analytical sample on the animal cells is derived from the result of at least one of the steps: second evaluation step A, C and/or third evaluation step A. This means that in a preferred embodiment, the cytotoxic effect of the analytical sample on the animal cells is derived directly from the degree of coverage. The third evaluation step A is not carried out in the procedure and the fourth evaluation step A, C is carried out after the second evaluation step A, C. This can bypass the determination of intermediate values and simplify the evaluation. In a further alternative preferred embodiment, the cytotoxic effect of the analytical sample on the animal cells is derived from the determined degree of proliferation from the third evaluation step A, i.e. indirectly from the degree of coverage. This allows the proliferative capability of the animal cells to be determined before the cytotoxic effect of the analytical sample is determined. This is particularly advantageous for graduated degrees of coverage and as intermediate information.

In a further alternative preferred embodiment, the cytotoxic effect of the analytical sample on the animal cells is determined from the determined degree of coverage in the second evaluation step A, C and from the proliferation capability in the third evaluation step A, both results being taken into account. The cytotoxic effect is preferably mapped in several levels. To derive the cytotoxic effect, the cultivation time can also be included.

Additionally, it is preferred that the animal cells are adherent growing animal cells. The method is particularly advantageous for adherently growing animal cells, as proliferation then proceeds adhesively on the surface of the sample spot. This increases the reliability of the method, as the adherent-growing animal cells are largely localized to the sample spot. Thus, the animal cells remain largely in place during the procedure. In addition, said cells already preferentially adhere to the surface during the cultivation step and proliferate there rather than in suspension. Adherent growing cells in suspension also do not form aggregates like suspension cells. Adhesion of the animal cells is also required for the sole determination of migratory capability.

According to a further advantageous embodiment, it is provided that the plurality of measuring positions are distributed locally in the at least one partial area of the sample spot in such a way that the determined degree of coverage is representative for the partial area and/or the sample spot. This increases the reliability of the procedures. Preferably, the plurality of measuring positions forms a uniform grid for this purpose. Preferably, in the uniform grid, each non-edge measuring position is equally spaced from the directly surrounding measuring positions in 4 orthogonal directions.

In a further preferred embodiment, the mass spectrometric sample in sample provision step A, B further comprises a potential cytotoxicity factor neutralizing factor. Thus, not only the effect of cytotoxic factors or analytical samples on the animal cells can be determined, but also a neutralizing effect of another factor on the cytotoxic factor or analytical sample can be determined. The cytotoxicity factor neutralizing factor may be selected from the following non-exhaustive group: Antibiotic, antimicrobial agent, antibacterial agent, antifungal agent, antiviral agent, antiparasitic agent, pharmaceutical agent, anti-virulence agent, antibody. Other cytotoxicity factor-neutralizing factors result depending on the application. In particular, cytotoxicity factor-neutralizing factors may be used in the method to assess the efficacy of the cytotoxicity factor-neutralizing factor against cytotoxic factors such as, for example, bacteria, fungi, parasites or viruses.

In order to derive the degree of proliferation and/or the cytotoxic effect, it is further advantageous that, in addition to the mass spectrometric sample comprising the analytical sample, at least one reference sample is processed by means of the method. In the process, the reference sample is measured and evaluated on a different sample spot using the method—analogous to the analytical sample. The mass spectrometric sample either comprises an analytical sample or is a reference sample. Preferably, the reference sample and the mass spectrometric sample comprising the analytical sample undergo the procedure simultaneously on the same mass spectrometric sample support and/or in direct sequence on different mass spectrometric sample supports. Preferably, the reference sample is free of cytotoxic factors and has the same animal cells and culture medium used in the mass spectrometric sample with the analytical sample. Preferred reference samples are samples with a known evaluation result, comprising: Degree of coverage, cell expansion distance, proliferation ability, migration ability and/or cytotoxic effect. This is to be distinguished from analytical samples, which concern samples to be examined with unknown evaluation results. Preferably, several reference samples are also analyzed on each mass spectrometric sample support.

Furthermore, after the determination of the degree of coverage in the second evaluation step A, C, a comparison of the degree of coverage of the reference sample with the degree of coverage of the mass spectrometric sample comprising the analytical sample is preferably carried out in an intermediate evaluation step A. Furthermore, preferably, after the determination of the cell expansion distance in the second evaluation step B, a comparison of the cell expansion distance of the reference sample with the cell expansion distance of the mass spectrometric sample comprising the analytical sample is carried out in an intermediate evaluation step B.

Thereby, the reference sample preferably does not comprise the at least one potentially cytotoxic factor, and/or the at least one cytotoxic factor and/or the at least one potentially cytotoxicity factor-neutralizing factor. Preferably, the reference sample has the same animal cells and culture medium used in the mass spectrometric sample with the analytical sample. This comparison is then preferably included in the derivation of the proliferation capability in the third evaluation step A, B and/or in the derivation of the cytotoxic effect in the fourth (preferably last) evaluation step. This ensures that the degree of coverage, cell expansion and/or proliferation is correctly assigned to a corresponding cytotoxic effect. This also ensures comparability between several process runs.

In another preferred embodiment, the mass spectrometric sample is provided in the sample preparation step A, B by having the animal cells already on the sample spots prior to application of the culture medium and the analytical sample. For example, the cells may be provided on the sample spot in a frozen or freeze-dried state. The mass spectrometric sample supports, for example, can already be delivered in this form by the manufacturer. This has the advantage that the user saves the application of the animal cells. Particularly preferably, the animal cells are already adhered to the sample spots before application of the culture medium and the analytical sample. This shortens the duration of cultivation in the cultivation step. Preferably, only part of the sample spot is covered with already adhered animal cells in sample preparation step A, so that proliferation can still be detected in the cultivation step afterwards. This means that the already adhered animal cells preferably cover the sample spot incompletely before application of the medium and the analytical sample. A degree of coverage in sample preparation step A between 10% and 90% of the area of the sample spot is advantageous. Furthermore, a degree of coverage in sample preparation step A between 25% and 75% of the area of the sample spot is preferred. A degree of coverage in sample preparation step A of between 30% and 50% of the area of the sample spot is particularly preferred. Preferably, the mass spectrometric sample support is delivered with animal cells already adhered to the sample spots. Preferably, cell-free positions are present in sufficient numbers to detect changes in the surface coverage of the sample spot with animal cells.

In an alternative preferred embodiment, the animal cells are provided by applying the animal cells to the sample spot in suspended form in sample provision step A, B. This has the advantage that the cells often grow better and the type of animal cells can be chosen. In certain applications, tissue cells from certain humans or patients can thus be also used as animal cells in the sample provision step A, B. For example, the effect of chemotherapeutic agents on tumor cells and healthy tissue cells from the same person can be tested directly. Furthermore, it is preferred that when the animal cells are applied in suspended form, a washing step is performed after the liquid removal step by washing the animal cells on the sample support and removing residual washing liquids. This ensures that only adhered cells are detected in measurement step A, B, C. Alternatively, the liquid removal step can be replaced by the washing step to shorten the process. Washing of the animal cells is usually done with buffer solution as washing liquid. For example, the washing liquid is applied and removed again. The amount of washing liquid is preferably chosen so that the sample spot is completely covered or the entire sample support is wetted. The removal of the washing liquid and/or residual liquid can be done, for example, by drying and/or suction/adsorption, for example by contact with an absorbent medium.

In another alternative preferred embodiment, the mass spectrometric sample is provided in the sample provision step A, B by having the animal cells already present on the sample spots, for example non-adhered, prior to application of the culture medium and the analytical sample. For example, the cells may be presented on the sample spot in a frozen or freeze-dried state. The mass spectrometric sample supports, for example, can already be delivered in this form by the manufacturer. This saves the end user from having to provide or add the animal cells themselves. After application of the remaining mass spectrometric sample (among others, the culture medium and the analytical sample), the preferred procedure is to proceed as described above for the application of the animal cells in suspended form.

According to an advantageous embodiment of the invention, the analytical sample is provided by providing source sample. This saves the effort of preparing the source sample. According to another advantageous embodiment of the invention, the analytical sample is provided by providing components of the source sample, for example soluble components. By a first purification or first separation of the source sample, in particular components of the source sample can be removed which would negatively influence the cultivation and/or the measuring step A, B, C. For example, in the case of stool samples, the removal of larger insoluble fragments such as undigested food residues would be relevant. Furthermore, compared to the complete isolation of the cytotoxic factor, the risk of the cytotoxic factor being depleted or altered during the isolation process is reduced. According to an alternative advantageous embodiment of the invention, the source sample is provided by providing the cytotoxic factor of the source sample in isolated and/or purified form. The cytotoxic factor may also have been isolated from the source sample. Isolation is more costly, but ensures that the procedure is carried out without interference.

Preferably, the sample of origin is a biological source sample. Furthermore, the source sample is preferably selected from the following group: Sample of a human, sample of an animal, sample of a plant or environmental sample. The sample from a human or animal may be, for example, a stool sample, urine sample, blood sample, whole blood, serum sample, plasma sample, cerebrospinal fluid sample, bronchoalveolar lavage (BAL) sample, wound secretion sample, other secretion and excretion samples, a swab from a wound or mucous membrane, a tissue sample, a muscle sample, a brain sample, a tumor sample, an organ sample or a skin sample, a soft tissue sample, a sputum sample, an excretion sample, or a punctate sample.

For example, the sample of a plant may be a sample of a potentially poisonous plant.

The environmental sample can be, for example, a water or wastewater sample, soil sample, air sample, surface sample, or a sample of a food, such as meat, milk, yoghurt or any other food.

In another advantageous embodiment, the animal cells are vertebrate cells, mammalian cells and/or human cells. Thus, the type of animal cells can be selected according to the application. The mass spectrum obtained and the cell-specific mass spectrometric signatures are usually each dependent on the type of animal cell used.

Human cells are used as animal cells in particular when the effect of a potentially cytotoxic factor on humans is to be tested. Human cells from a particular person are particularly preferred if a cytotoxic effect is to be investigated specifically for that person. For example, the cytotoxic effect of chemotherapeutic agents on tumor cells and healthy tissue cells of a human can be tested to check the effect of the chemotherapeutic agent before administration. The animal cells can also be nerve cells, especially human nerve cells. This makes it particularly advantageous, for example, to check for the presence of neurotoxins as cytotoxic factors. The use of vertebrate cells, such as amphibian cells or fish cells, for example, enables sensitive testing of environmental toxins.

Furthermore, it is advantageous if the animal cells are continuous cell lines, in particular continuous human cell lines. Continuous cell lines are particularly easy to cultivate. Most currently available cell culture lines are mammalian cell lines. Examples include MDCK, HeLa, HECK, PERC and CHO cell lines. The ease of cultivation thereof makes said cells suitable, for example, for use in broad screening procedures, such as the search for new pharmacological agents. In another preferred embodiment, the animal cells are derived from tissue samples taken from a human or animal. Furthermore, the animal cells preferably originate from tumor samples taken from a human or animal. This is a simple way to determine the cytotoxic effect of drugs on this human or animal before administering them.

According to an advantageous embodiment of the invention, the at least one cytotoxic factor is from the following group: chemical elements including isotopes and ions thereof, such as certain metals, low molecular weight chemical compound including ions thereof, high molecular weight chemical compound, pharmaceutical agent, toxin, archaea, bacterium, virus, fungus, protozoan, algae, parasite, components or secreted substances of microorganisms and other transmissible biological agents, cytotoxic substances of biological origin, cytotoxic substances of microbial origin from a human sample, chemotherapeutic agent, antitumor agent, protein, peptide, antibody, antimicrobial agent, antiviral agent, antifungal agent, antiparasitic agent or antibacterial agent or biocide. The microorganisms here include in particular archaea, bacteria, viruses, unicellular fungi, unicellular algae, protozoa and/or other unicellular parasites. It should unequivocally be made clear that the above members of the cytotoxic factor group represent the respective type of cytotoxic factor, but do not limit the quantity or diversity. The respective cytotoxic factors are preferably also the multiplicity of a single unit. For example, the cytotoxic factor bacterium is a large number of bacterial cells. Furthermore, the bacterial cells can belong to one or more bacterial strains. Furthermore, for example, a variety of archaeal cells (archaea), viral particles (virus), fungal cells (fungus), protozoan cells, algal cells, parasitic organisms (parasite), protein molecules (protein), and so on is also included. All listed organisms (microorganisms, parasites) can be dead or alive. Furthermore, components and/or secreted substances of all listed organisms and viruses are also included. Parasites are particularly favored single-celled parasites such as protozoa. Further preferably, the parasites also comprise components and/or secreted components of multicellular parasites, such as parasitic worms. The group of cytotoxic factors thus preferably ranges from chemical compounds to viruses and components of organisms to microorganisms or other transmissible biological agents. The broad applicability is a particular advantage of the claimed process. Various cytotoxic factors from this group may also be present in combination in the mass spectrometric sample.

Particularly preferably, the analytical sample comprises, as cytotoxic factor, bacterial cells, for example of one or more bacterial strains, or components thereof, and, as cytotoxicity factor-neutralizing factor, an antibacterial agent which is directed against bacterial cells or components thereof and potentially has an antibacterial effect on bacterial cells or components thereof. Thus, the effect of an antibacterial agent on bacterial cells or components thereof can be tested in a simple way. If the cytotoxic effect of the bacterial cells or components thereof has been previously determined or is known, an unaffected proliferative capability of the animal cells in the presence of the bacterial cells or components thereof and the antibacterial agent indicates an antibacterial effect of the antibacterial agent against these bacterial cells (bacterial strain—s). On the other hand, a reduced proliferation capability of the animal cells in the presence of the bacterial cells or components thereof and the antibacterial agent indicates a (partial) resistance of the bacterial cells (bacterial strain—s) to the antibacterial agent. For this purpose, the mode of action or structure of the antibacterial agent need not be known, as only the cytotoxicity factor-neutralizing effect is detected here, which is an advantage when screening for new antibacterial agents. At the same time, the cytotoxic effect of the antibacterial agent on the animal cells can be determined. This has the advantage that the same procedure can be used to select antibacterial agents that act specifically on the bacteria in question. The risk of a harmful effect on a human can be further reduced if the animal cells are human cells. Components of bacterial cells also include substances secreted by the bacterial cells.

Furthermore, it is not necessary to know which cytotoxic factor an analytical sample contains. Thus, with these methods, various cytotoxicity factor-neutralizing factors can be tested safely using the analytical sample—instead of on living humans or animals.

In a further preferred embodiment, the analytical sample comprises as cytotoxic factor virus particles, for example of one or more virus strains, or components thereof and as cytotoxicity factor neutralizing factor an antiviral agent directed against the virus particles or components thereof. Thus, the effect of an antiviral agent on the virus particles or components thereof can be easily checked. If the cytotoxic effect of the virus particles or components thereof has been previously determined or is known, an unaffected proliferative capability of the animal cells in the presence of virus particles or components thereof and the antiviral agent indicates an antiviral effect of the antiviral agent against the virus (virus strain—s). On the other hand, a reduced proliferation capability of the animal cells in the presence of the virus particles or components thereof and the antiviral agent indicates a (partial) resistance of the virus (virus strain—s) to the antiviral agent. For this purpose, the mode of action or structure of the antiviral agent does not need to be known, as only the effect is demonstrated here, which is a great advantage when screening for new antiviral agents. At the same time, the cytotoxic effect of the antiviral agent on the animal cells can be determined. This has the advantage that only antiviral agents that specifically act on the respective viruses (virus strain—s) can be selected. The risk of a harmful effect on a human can be further reduced if the animal cells are human cells.

In an alternative advantageous embodiment, the analytical sample comprises as cytotoxic factor fungal cells (comprising fungal strain—s) or components thereof and as cytotoxicity factor-neutralizing factor an antifungal agent directed against the fungal cells or components thereof. Thus, the effect of an antifungal agent on the fungal cells or components thereof can also be tested in a simple way. If the cytotoxic effect of the fungal cells or components thereof has been previously determined or is known, an unaffected proliferative capability of the animal cells in the presence of the fungal cells or components thereof and the antifungal agent indicates an antifungal effect of the antifungal agent against the fungal cells or components thereof. On the other hand, a reduced proliferation capability of the animal cells in the presence of the fungal cells or components thereof and the antifungal agent indicates a (partial) resistance of the fungus to the antifungal agent. For this purpose, the mode of action or structure of the antifungal agent need not be known, as only the effect is demonstrated here, which is a great advantage when screening for new antiviral agents. At the same time, the cytotoxic effect of the antifungal agent on the animal cells can be determined. This has the advantage that only antifungal agents that specifically act on the respective fungus can be selected. The risk of a harmful effect on a human can be further reduced if the animal cells are human cells. Components of fungal cells also include substances secreted by the fungal cells.

In an alternative advantageous embodiment, the analytical sample comprises as cytotoxic factors at least one parasite or components thereof and as cytotoxicity factor-neutralizing factor an antiparasitic agent directed against the parasite or components thereof. Thus, the effect of an antiparasitic agent on the parasite or components thereof can also be easily checked. If the cytotoxic effect of the parasite or components thereof has been previously determined or is known, an unaffected proliferative capability of animal cells in the presence of the parasite or components thereof and the antiparasitic agent indicates an antiparasitic effect of the antiparasitic agent against the parasite. On the other hand, a reduced proliferative capability of the animal cells in the presence of the parasite or components thereof and the antifungal agent indicates a (partial) resistance of the parasite to the antiparasitic agent. For this purpose, the mode of action or structure of the antiparasitic agent need not be known, as only the effect is demonstrated here, which is a great advantage when screening for new antiparasitic agents. At the same time, the cytotoxic effect of the antiparasitic agent on the animal cells can be determined. This has the advantage that only antiparasitic agents can be selected that specifically act on the respective parasite. The risk of a harmful effect on a human can be further reduced if the animal cells are human cells. Components of parasites also include substances secreted by the parasites.

In order to determine a concentration-dependent cytotoxic effect of the analytical sample, it is further advantageous that different concentrations of the analytical sample are provided to different sample spots in each case. The concentration-dependent cytotoxic effect is preferably a function of the determined degrees of coverage (claim 1, 2) or the determined cell expansion distances (claim 3) of the different sample spots and the respective different concentrations of the analytical sample. This makes it easy to determine the concentrations of the analytical sample at which cell toxicity occurs. This can be used, for example, in the investigation of active pharmaceutical ingredients such as antibiotics or chemotherapeutics for the initial assessment of an applicable concentration range.

In addition, it is preferred that the spatial resolution mass spectrometer and/or the mass spectrometer is selected from the following group: Laser Desorption Ionization-MS (LDI-MS), Matrix Assisted Laser Desorption/Ionization—MS (MALDI-MS), Desorption Electrospray Ionization-MS (DESI-MS), Matrix Assisted Laser Desorption Electrospray Ionization-MS (MALDESI-MS), Secondary Ions-MS (SIMS), Secondary Ion Mass Spectrometry Imaging-MS (SIMS-Imaging-MS), Matrix-assisted laser desorption/ionization time-of-flight analyzer-MS (MALDI-ToF-MS), MALDI tandem time-of-flight mass analyzer—MS (MALDI-ToF-ToF-MS), MALDI mass spectrometry imaging (MALDI-MSI) or spatially resolved MS with a micro-fluid sample ionization device.

The spatial resolution mass spectrometers can be further subdivided into matrix-based and non-matrix-based spatial resolution mass spectrometers. In matrix-based spatial resolution mass spectrometers, a matrix, preferably liquid, is added to the mass spectrometric sample. This allows the analytes of the mass spectrometric sample to be ionized during laser bombardment. The generated sample ions are then measured. In non-matrix-based spatial resolution mass spectrometers, on the other hand, no matrix is added. A matrix is not required here to ionize the analytes of the mass spectrometric sample and to generate sample ions from the mass spectrometric sample.

For example, matrix-based spatial resolution mass spectrometers preferably includes the following MS: MALDI-MS, MALDESI-MS, MALDI-ToF-MS, MALDI-ToF-ToF-MS, MALDI-MSI. Non-matrix based spatial resolution mass spectrometers preferably include, for example, the following spatial resolution mass spectrometers: LDI-MS, DESI-MS, SIMS, SIMS imaging and the spatial resolution mass spectrometer with a micro-fluid sample ionization device. Furthermore, all combinations with ion mobility analyzers are included in the preferred embodiments.

In a further preferred embodiment, the true-to-position application of the matrix is conducted by means of a spraying process, sublimation process or sequential directional positioning of a plurality of matrix microdroplets or matrix nanodroplets on the sample spot. While the application of the matrix by means of a spraying process and sublimation processes is largely untargeted, the application by means of sequential directional positioning is usually targeted. Due to the simplicity and speed of the process, the application of the matrix is particularly preferred using a spray method. The matrix is applied by spraying the sample spots with matrix or the mass spectrometric sample support using a matrix spraying device. The matrix is preferably applied micro dispersed. The matrix spraying device produces smallest matrix droplets: Matrix microdroplets and/or matrix nanodroplets. Preferably, the applied matrix droplets have a diameter of 0.01 to 100 μm. Preferably, the applied matrix droplets have a diameter of 0.05 to 40 μm. Particularly preferably, the applied matrix droplets have a diameter of 0.1 to 10 μm. Further procedures and details for the true-to-position application of the matrix are known to the person skilled in the art in the context of sample preparation for imaging mass spectrometry.

The task is further solved with a further method for determining the cytotoxic effect of an analytical sample on animal cells, comprising the following steps:
a sample provision step A in which a mass spectrometric sample comprising animal cells, culture medium, and the analytical sample potentially having a cytotoxic factor is provided on at least one sample spot of a mass spectrometric sample support;
a cultivation step in which the mass spectrometric sample is incubated on the sample spot of the mass spectrometric sample support in a cultivation device;
a liquid removal step, in which residual liquid of the mass spectrometric sample is removed from the sample spot;
a measuring step C, in which mass spectra are recorded at a plurality of measuring positions distributed over at least a partial area of the sample spot by means of a mass spectrometer, wherein, when a matrix-based mass spectrometer is used, the sample spot is prepared in a preceding sample preparation step A by a true-to-position application of a matrix to at least the one partial area of the sample spot;
a first evaluation step C in which each mass spectrum is analyzed for the presence of a cell-specific mass spectrometric signature for the animal cells;
a second evaluation step C, in which a degree of coverage by animal cells is determined for at least the partial area of the sample spot from the result of the first evaluation step C;
a fourth evaluation step C, in which the cytotoxic effect of the analytical sample on the animal cells is derived from the determined degree of coverage of the second evaluation step C.

It has been shown that the determination of the degree of coverage and the cytotoxic effect of a sample that can be derived from it is also possible by means of a method that does not use a spatial resolution mass spectrometer. With this method, no exact location coordinates of the measuring positions are required. Thus, the mass spectra generated are not spatially resolved. However, the individual mass spectra of the measuring positions of a sample spot are not calculated into a sum mass spectrum in this procedure either. The measuring positions are distributed over at least a partial area of the sample spot. The measuring positions are largely randomly distributed. Preferably, said positions are largely evenly distributed. The degree of coverage can thus be determined by a correspondingly high number of measuring positions. Preferably, the diameter of the laser beam/desorption beam and/or the sample point is known. In this procedure, the degree of coverage represents a statistical average value. The quantity of measuring positions to be measured is preferably so high that a representative degree of coverage can be determined which is largely reproducible and has sufficient accuracy, for example in the form of a statistically acceptable standard deviation. This method has the advantage that it does not require a spatial resolution mass spectrometer and it is particularly cost-effective. All that is needed is a mass spectrometer that can measure various numerous measuring positions on a sample spot. Thereby the precise adjustment of the measuring position, for example by means of a precise X/Y table and/or the precise adjustment of mirrors is not necessary. An approximate approach to each measuring position is sufficient as long as a large number of the numerous measuring positions of a sample spot differ. However, a disadvantage compared to the other methods is a reduced accuracy and/or reproducibility of the determined degree of coverage depending on the number of measuring positions and a corresponding increase in the time required per sample position. In addition, the diameter of the laser or desorption beam is preferably smaller than in the other methods, as a higher number of measuring positions is required. Preferably, the diameter of the laser or desorption beam or of the measuring position is selected in such a way that, with the required number of measuring positions, there is largely no mathematical overlap of the measuring positions. Preferably, a cell presence value is assigned to each measuring position. Preferably, the degree of coverage by animal cells is determined for at least the partial area of the sample spot from the cell presence values of the measuring positions. Furthermore, in a third evaluation step A, a proliferation capability is derived from the determined degree of coverage. Further preferably, the cytotoxic effect of the analytical sample on the animal cells is derived from the degree of coverage and/or the proliferative capability.

The task is further solved with a method for determining the cytotoxic effect of at least one analytical sample on animal cells comprising the following steps:
a sample provision step B in which a mass spectrometric sample comprising animal cells, nutrient medium, and said analytical sample potentially having a cytotoxic factor is provided on at least one sample spot of a mass spectrometric sample support, said sample spot having a first and a second partial area, the animal cells being provided on said first partial area of said sample spot and no animal cells being provided on said second partial area of said sample spot, and the remaining mass spectrometric sample being provided on both partial areas;
a cultivation step in which the mass spectrometric sample is incubated on the sample spot of the mass spectrometric sample support in a cultivation device;
a liquid removal step in which residual liquids of the mass spectrometric sample on the sample spot are removed;
a measuring step B in which spatially resolved mass spectra are recorded at at least two measuring positions of the sample spot by means of a spatial resolution mass spectrometer, the first measuring position still having animal cells and the second measuring position having no animal cells,
   wherein both measuring positions are located on one direction of extension starting from a reference point, wherein, when using a matrix-based spatial resolution mass spectrometer, prior to the recording of the spatially resolved mass spectra in the measuring step B, in a preceding sample preparation step B a preparation of the sample spot is carried out by a true-to-position application of a matrix to at least a part of both partial areas of the sample spot;
a first evaluation step A in which each spatially resolved mass spectrum is analyzed for the presence of a cell-specific mass spectrometric signature for the animal cells and a cell presence value is assigned to each measuring position;
a second evaluation step B in which a cell expansion distance is determined from at least one distance from a reference point to a transition point, wherein each transition point is calculated from at least the first and second measuring positions;
a fourth evaluation step B, in which the cytotoxic effect of the analytical sample is derived from the result of the preceding evaluation step.

This allows the presence of cytotoxic factors in an analytical sample, of which it is not known whether said sample contains one or more cytotoxic factors, to be checked reliably, simply, quickly and inexpensively. In addition, the effect of potentially cytotoxic factors on animal cells can be tested and the toxicity thereof determined reliably, simply, quickly and inexpensively.

It has been shown that for the determination of the cytotoxic effect of analytical samples, the determination of the cell expansion distance, and/or the proliferation ability and/or migration ability derived from this, by determining the expansion of the animal cells on the sample spot by means of spatial resolution mass spectrometers can also be used. Preferably, the expansion of the animal cells on the sample spot is determined in this method by determining a cell expansion distance. According to claim 3, the preferred result of the immediately preceding evaluation step is the cell expansion distance and the preferred preceding evaluation step is the second evaluation step B.

Alternatively, according to claim 3, the result of the preceding evaluation step is the proliferation capability and/or the migration capability and the preferred preceding evaluation step is the third evaluation step B. The additional process steps provide the user, among others, with further data for the purpose of better result evaluation.

It is decisive for the method according to claim 3 that the cell expansion distance of the animal cells starts in the form of a two-dimensional spreading from the outer edge of the first partial area by growth, cell division and/or cell migration under conditions suitable for proliferation and/or cell migration. It is crucial that the second part of the sample spot, which is adjacent to the first part of the sample spot, is free of animal cells at the beginning of the cultivation step. The cytotoxic effect is ultimately determined by the extent of the expansion of the animal cells. For example, the radial extent of the coverage of the second subarea with animal cells by proliferation and/or migration is a measure of the cytotoxic effect of an analytical sample or a cytological factor.

In a simple way, the extent of the expansion of the animal cells can be achieved by determining the cell expansion distance. The boundary of the expansion area of the animal cells at a time t is determined. The determination of the boundary of the expansion area is done by determining an transition point. The determination of the transition point is done by detecting the presence or absence of the animal cells at the boundary by mass spectrometry. The cell expansion distance is then determined by determining the distance from a reference point to the transition point.

By providing the animal cells on a first partial area and not providing animal cells on a second partial area in sample provision step B, the extent of the animal cells can be used as a reliable measure of proliferative capability, migratory capability and/or cytotoxic effect. Preferably, the animal cells are adherent growing cells. Furthermore, preferably, the animal cells are already adhered to the first partial area before the addition of the remaining mass spectrometric sample. The sample provision step B is divided into a first and a second sub-step. Thus, in a first sub-step of the sample provision step B, a first cultivation of animal cells with nutrient medium takes place on the first partial area of the sample spot. An analytical sample or cytotoxic factor is not present here. In this process, the animal cells adhere to the first partial area and proliferate on the first partial area. The second partial area remains free of animal cells. This can be done, for example, through the targeted application of nutrient medium. After the first sub-step of sample provision step B, the residual liquid is preferably removed from the first partial area. After the first sub-step, the second sub-step of sample provision step B takes place. In the second sub-step of sample provision step B, nutrient medium, the analytical sample and further components of the mass spectrometric sample are applied to both partial areas or the entire sample surface. No animal cells are applied here.

In the cultivation step, a two-dimensional spreading takes place from the outer edge of the first partial area. This is preferably done largely uniformly in the cultivation step. In this case, the expansion preferably takes place in both dimensions of the surface. In particular, a circular design of the first partial area with surrounding second partial area results in a two-dimensional spread. Alternatively, the first partial area and the second partial area can be designed in such a way that the expansion is largely one-dimensional, i.e. in one direction. Further preferably, the sample spot is washed with a wash solution after the first and/or second sub-step of the sample provision step B and/or after the cultivation step in order to remove non-adhered animal cells.

The first partial area can be a circular surface which is completely surrounded by the second partial area in the form of a ring. Alternatively, the first partial area may not be fully adjacent to the second partial area. The decisive factor is that both partial areas are adjacent to each other.

The cultivation step and the liquid removal step are carried out as previously described.

By recording spatially resolved mass spectra at least two measuring positions of the sample spot in measuring step B using a spatial resolution mass spectrometer, the expansion boundary of the animal cells is determined. The expansion boundary is determined directly with the help of the transition point. The first measuring position still has animal cells. The second measuring position does not have any animal cells. Thus, the first measuring position and the second measuring position are each on a different side of the expansion boundary of the animal cells. Both measuring positions are preferably located directly at the expansion boundary. Preferably, the two measuring positions are adjacent. The closer both points are located to the expansion boundary, the more precisely the cell expansion distance can be determined. By means of a preferred multiple measurement along at least one expansion direction of the animal cells each, a first measuring position and a second measuring position can be found having the greatest proximity to the expansion boundary. Preferably, the second measuring position is located on the second partial area.

The preparation of the sample spot by a true-to-position application of a matrix to at least a partial area of the sample spot using a matrix-based spatial resolution mass spectrometer prior to the recording of the spatially resolved mass spectra in measuring step B in a preceding sample preparation step B has already been described before in an analogous manner within the framework of sample preparation step A, whereby here, in contrast, the application of the matrix to at least a part of both partial areas of the sample spot takes place. Particularly preferably, the matrix is applied to the entire sample spot. Alternatively, an application of the matrix can be applied to a part of both partial areas of the sample spot in an expansion direction starting from the reference point. When using non-matrix based spatial resolution mass spectrometers, sample preparation step B is not performed in the method.

Likewise, the first evaluation step A has already been described previously.

In the second evaluation step B, the cell expansion distance is determined. This is determined by means of the distance from a reference point to a transition point. In the process, several distances of a sample spot can also be calculated into one cell expansion distance. For example, distances along different directions of expansion can be used for this purpose. Each transition point is calculated at least from the first and the second measuring position with different cell presence values. The first measuring position preferably has a cell presence value which indicates the presence of an animal cell. The second measuring position preferably shows a cell presence value which indicates the absence of an animal cell. Preferably, one or further measuring positions, in the direction of extension starting from the reference point, in front of the first measuring position, essentially comprise cell presence values which indicate the presence of animal cells. Furthermore, one or further measuring positions, in the direction of extension starting from the reference point, behind the second measuring position, essentially comprise cell presence values which indicate the absence of animal cells. This allows the transition point to be determined precisely.

In the fourth evaluation step B, the cytotoxic effect of the analytical sample or the cytotoxic factor on the animal cells is preferably derived from the cell expansion distance. Preferably, the cell expansion distance is compared with stored reference values or with the values of reference samples. Preferably, at least one reference sample passes through the method in addition to the mass spectrometric sample comprising the analytical sample. Alternatively, stored reference values from reference samples can be used. For the possible different types of reference samples, please refer to the explanations on reference samples in the description.

In a further preferred embodiment, the method according to claim 3 comprises a third evaluation step B, in which a proliferation ability and/or a migration ability of the animal cells is derived from the determined cell expansion distance of the second evaluation step B. Here, the cytotoxic effect of the analytical sample or the cytotoxic factor is derived in the fourth evaluation step B from the determined proliferation ability and/or the migration ability of the animal cells.

Thus, the cytotoxic effect is derived either directly from the cell expansion distance or indirectly from the cell expansion distance via the proliferation ability and/or migration ability. The direct derivation simplifies the procedure. In the direct derivation, for example, several cell expansion distances of a sample spot or several sample spots can also be offset against each other. In particular, averaging can take place. The indirect derivation of the cytotoxic effect from the cell expansion distance provides the user with further useful intermediate information. Determining proliferative capability and/or migratory capability is particularly useful for graded cell expansion ranges. Furthermore, several cell expansion ranges can also be offset against each other to form a proliferation capability and/or migration capability. In a further alternative embodiment, the cytotoxic effect of the analytical sample on the animal cells can be determined from the determined cell expansion distance in the second evaluation step B and from the proliferation ability and/or migration ability in the third evaluation step B, taking both results into account.

The cytotoxic effect is preferably mapped in several levels.

In a further preferred embodiment, the method according to claim 3 comprises an intermediate evaluation step B. In the intermediate evaluation step B, a comparison of the cell expansion distance of at least one reference sample with the cell expansion distance of the mass spectrometric sample comprising the analytical sample is performed. This comparison can be included in the derivation of proliferative ability and/or migratory ability in third evaluation step B and/or in the derivation of cytotoxic effect in fourth evaluation step B. In this way, the cytotoxic effect of the analytical sample can be reliably inferred from the cell expansion distance in a simple manner. In an alternative preferred embodiment, the comparison is performed with stored reference values.

The problem is further solved with a system for determining the cytotoxic effect of an analytical sample on animal cells by means of a spatial resolution mass spectrometer comprising at least one spatial resolution mass spectrometer for generating spatially resolved mass spectra, a mass spectrometric sample support having sample spots, and a data processing unit for controlling the spatial resolution mass spectrometer and for evaluating the spatially resolved mass spectra generated and being configured to analyze each spatially resolved mass spectrum with respect to the presence of at least one cell-specific mass spectrometric signature for the animal cells; to determine a degree of coverage by the animal cells for at least a partial area of the sample spots and/or a cell expansion distance of the animal cells in at least one direction of extension of the sample spot; and to derive the cytotoxic effect of the analytical sample from the degree of coverage and/or from the cell expansion distance.

In this context, the cytotoxic effect can preferably be derived directly from the degree of coverage and/or the cell expansion distance, or indirectly via a proliferation capability and/or migration capability determined from the degree of coverage and/or the cell expansion distance. Directly explicitly includes other simple standard mathematical calculations, such as averaging or comparison with a database.

The spatial resolution mass spectrometer is preferably a mass spectrometer from the following group: LDI-MS, MALDI-MS, DESI-MS, MALDESI-MS, SIMS, SIMS imaging-MS, MALDI-ToF-MS, MALDI-ToF-ToF-MS, MALDI-MSI, spatial resolution mass spectrometer with a micro-fluid sample ionization device. The data processing unit is preferably a computer, which is integrated in the mass spectrometer or set up as a separate system. In addition, the computer preferably includes appropriate software with algorithms for evaluating and controlling the mass spectrometer. The evaluation preferably comprises the evaluation steps of the procedures described above. Also, the data processing unit preferably controls the spatial resolution mass spectrometer to perform the measurement step A, B, C of the aforementioned procedures. In particular, the software together with the data processing unit preferably performs calculations for the procedures described above. For example, the value of the reference sample can also be calculated with the value of the mass spectrometric sample using the software. Furthermore, the software with the data processing unit preferably displays the results. Preferably, information on the cytotoxic effect in a mass spectrometric sample is output for each sample spot. Alternatively, or additionally, the proliferation ability and/or the migration ability is preferably indicated for each sample spot. Preferably, the system is adapted to perform the methods according to claims 1, 2 and/or 3 and/or the dependent claims. Alternatively, instead of a spatial resolution mass spectrometer, the system may comprise a non-spatial resolution mass spectrometer when adapted to perform the method of claim 3.

Since the methods according to the disclosure are suitable as modes of operation of the system according to the disclosure, the embodiments explained above with reference to the methods are also applicable to the system in a corresponding manner.

Similarly, the embodiments and explanations of the features, such as the degree of coverage and/or proliferative capability, explained with reference to the processes according to claim 1 and its subclaims, are fully or partially applicable to the processes according to claim 2 and/or 3, with corresponding transferability obvious to the person skilled in the art.

The range of applications of the procedures and the system is wide. In the following, the term “process” will be used uniformly with regard to the possible applications, thus including all claimed processes and the system. The method can be used in the clinical field, among others. The result of the procedure can be supportive for diagnoses. Virulence can be directly inferred from the cytotoxic effect in the procedure. In particular, the presence of a virulent pathogen can be inferred from the cytotoxic effect. Particularly advantageous is the combination of the method according to the invention with other analytical methods which can detect the presence of a cytotoxic factor but not the cytotoxic effect thereof.

For example, despite the lack of information on the exact mechanism of resistance of, for example, viruses (cytotoxic factors) to an antiviral agent (cytotoxic factor-neutralizing factors), the method can indicate the resistance of a virus to an antiviral agent. For this purpose, continuous cell lines can be used as animal cells, for example. Examples include viral infections with or outbreaks of the viruses herpes simplex virus (HSV), varicella zoster virus (VZV) or cytomegalovirus (CMV).

In particular, the combination of a microorganism identification software module (for example Bruker's MBT Compass including the Library) with the method can not only detect the presence of a cytotoxic factor, but in addition to the genus and/or species of a microorganism by means of, for example, a MALDI biotyper, a more accurate classification, in particular of virulence phenotypes, can be made. In certain cases, the taxonomy level subspecies and/or the taxonomy level pathovar can thus also be determined with the method. This can, for example, speed up and/or specify a diagnosis or improve clinic management. The following are some examples of clinical applications. For example, the presence of hypervirulent strains of Klebsiella pneumoniae can be detected easily and quickly with the method. Furthermore, the method can be used, for example, to detect the presence of relevant virulence phenotypes of uropathogenic Escherichia coli strains easily and quickly in urine. The method is also suitable, for example, for the detection of Clostridioides difficile and/or the toxin thereof in severe diarrheal diseases. Thus, the method offers a fast and reliable identification of C. difficile-associated diarrhea. Furthermore, the method is also suitable, for example, for the detection of diarrhea toxin-producing strains of Bacillus cereus after prior determination of the species Bacillus cereus using a microorganism identification software module.

In particular, the method is also suitable for the detection of virulent pathogens, whose virulence is only given under certain conditions and is not solely determined by the presence of the pathogens. For example, the method is suitable for distinguishing between a very acute course of endocarditis due to a highly virulent Staphylococcus aureus strain and a low-acuity course of endocarditis due to a weakly virulent Staphylococcus aureus strain. Furthermore, the method is suitable for easily detecting the presence of cytotoxic factors such as botulinum neurotoxins in analytical samples using nerve cells as animal cells. Furthermore, when using tumor cells as animal cells and chemotherapeutic agents as cytotoxic factors, the method is suitable for easily determining the sensitivity of tumor cells to chemotherapeutic agents for estimating the therapeutic efficacy of chemotherapy.

### Definitions

The spatially resolved mass spectrum comprises the raw mass spectrometric data up to a processed peak list that only shows the intensities of the respective mass ranges (m/z) and the associated measuring positions. A spatially resolved mass spectrum can consist of a plurality of intensity values in a coherent mass range as well as of the intensity values of several separate mass ranges. The spatially resolved mass spectrum may be subjected to signal processing prior to use in the method, including, for example, baseline correction (subtraction), smoothing of mass signals, elimination of noise signals, selection of mass signals above a specified noise value, and/or peak finding and/or feature detection to produce a peak list. The spatially resolved mass spectrum is preferably a single mass spectrum. Each single mass spectrum is preferably generated by one single laser or beam bombardment. The spatially resolved mass spectrum can be a sum mass spectrum of one measuring position, insofar as individual mass spectra of the same measuring position were combined. However, a spatially resolved mass spectrum in the sense of the invention is not a sum mass spectrum of the sample spot, i.e. the individual mass spectra of different measuring positions of the sample spot are not combined. Preferred m/z ranges are between m/z 1000 and m/z 20,000, further preferred between m/z 2000 and m/z 15,000, especially preferred between m/z 2000 and m/z 10,000.
The term method is preferably used for the entire invention if a more detailed specification of the method is not apparent or obvious to the person skilled in the art from the context. Thus, if a more detailed specification of the method is not apparent or obvious from the context, the term method in non-specifiable form preferably refers to part or all of the claims (including system claim)—as far as applicable according to the person skilled in the art.
Source sample is an extracted sample. Preferably, said sample is largely unchanged since extraction.
Cytotoxicity and the cytotoxic effect are the measure of the ability of certain cytotoxic factors to damage animal cells, such as immune cells. It can be concentration dependent. The cytotoxic effect on animal cells may be proliferation slowing and/or proliferation inhibiting. Furthermore, the cytotoxic effect on animal cells may be growth inhibiting and/or growth retarding. Furthermore, the cytotoxic effect on animal cells may be cell division inhibiting and/or cell division slowing. Furthermore, the cytotoxic effect on animal cells can be necrotic. Furthermore, the cytotoxic effect on animal cells may be cell migration inhibiting and/or cell migration slowing.
Cytotoxic factors are factors with (potential) cytotoxic effects. Said factors can slow down or stop the proliferation, cell migration, division and/or growth of the animal cells or destroy the animal cells. Cytotoxic factors include any chemical substance or biological matter, in particular bacteria, viruses, fungi, micro-organisms, parasites, dead or alive, active or inactive, having an actual or potential cytotoxic effect on at least one type of animal cell. Said factors can be present, for example, dissolved, suspended, or adsorbed or immobilized on a surface (for example, on the surface of the mass spectrometric sample support). Environmental conditions such as temperature or atmospheric air composition are not cytotoxic factors.
Animal cells are cells of organisms from the taxonomic level: Kingdom: Animals. The group of animals within the definition of “animal cells” includes, but is not limited to, cells of insects, such as arthropods and/or annelids; vertebrates, mammals, and/or humans. Protozoa and/or Euglena are preferably excluded from the animal cells. Furthermore, the animal cells are preferably tissue-forming cells. The animal cells are preferably not part of the analytical sample. The animal cells can be, for example, samples of tissue cells or tumor cells from humans or animals, especially mammals. In particular, said cells can be continuous cell lines. The animal cells are, in the context of the application, animal cells whose proliferative capability and/or migratory capability is used to determine the cytotoxic effect of a cytotoxic factor or analytical sample.
Microorganisms preferably include all prokaryotic organisms, including Archaea bacteria and Bacteria. Furthermore, microorganisms include all protozoa. Furthermore, microorganisms include certain eukaryotic organisms, such as certain (especially unicellular) fungi, such as yeast, and (especially unicellular) algae. Tissue-forming eukaryotic organisms do not count as microorganisms. Preferably, eukaryotic microorganisms are unicellular. Furthermore, microorganisms include viruses. Microorganisms may preferentially colonize and/or infect and/or be present on and/or in animal, including human, plant or microbial hosts or host cells. Microorganisms also preferably include microorganisms that occur independently of hosts in the environment (e.g., water, soil, air, surfaces). The term microbial is equivalent to the term microorganism.
Sample spot is preferably a sub-area of the mass spectrometric sample support on which a mass spectrometric sample or reference sample is applied and measured. Furthermore, the sample spot is preferably a sub-area of the mass spectrometric sample support which is intended to receive and/or cultivate a mass spectrometric sample.
Residual liquid: The liquids of all components of the mass spectrometric sample together are the residual liquid. Cell contents of intact animal cells are not part of the residual fluid.
Expansion and areal spreading of the animal cells preferably mean the areal spreading of the animal cell population caused by proliferation, growth and/or division of the animal cells. Alternatively, or in addition to proliferation, growth and/or division, the planar spreading in the context of the expansion and planar expansion of the animal cells can also be caused by cell migration of the animal cells.
Degree of confluence is the degree of coverage of the surface of a sample spot with adherently growing animal cells. Adherent-growing animal cells are here synonymous with the term adherent-proliferating animal cells.
Analytical samples include samples to be tested with unknown evaluation result, namely the potential presence of a cytotoxic effect on the animal cells. Thus, a solution with known cytotoxic substances also counts as an analytical sample as long as the evaluation result is unclear. For example, a solution with a substance classified as cytotoxic in different concentrations and/or in combination with different tissue cells as animal cells is also to be classified as an analytical sample. The analytical sample is a sample that is analyzed with the spatial resolution mass spectrometer. It potentially includes at least one cytotoxic factor. The analytical sample may be provided at the sample spot in an unaltered state, i.e. for example as an source sample, or it may preferably be subjected to an enrichment or culture preparation, concentration and/or extraction or purification step according to methods known to the person skilled in the art to concentrate, isolate or purify the potential cytotoxic factor before being provided at the sample spot.
Reference samples are used to generate reference values. Reference samples are preferably samples with known and/or expected evaluation results. Preferably, said samples run through the procedure in parallel with the mass spectrometric sample comprising the analytical sample with unknown evaluation result. Alternatively, the reference values are generated in advance, stored in a database, and retrieved when the claimed process is carried out. For a better understanding, examples of reference samples are given below. The reference samples can preferably be selected from the following group:
   Blank sample; only the matrix used, if a matrix is used in the procedure, is measured
   Mass spectrometric sample consisting of nutrient solution
   Mass spectrometric sample consisting of animal cells
   Mass spectrometric sample consisting of nutrient solution and animal cells
   Mass spectrometric sample consisting of one or more cytotoxic factors
   Mass spectrometric sample consisting of one or more cytotoxicity factor-neutralizing factors
   Mass spectrometric sample includes nutrient solution, animal cells and one or more cytotoxic factors with known cytotoxic effect on the animal cells used and thus known evaluation result.
Thus, the reference samples serve, among other things, to assign the mass ranges to components of the mass spectrometric sample. This allows cell-specific and non-cell-specific mass spectrometric signals to be differentiated and cell-specific mass spectrometric signatures to be identified. Furthermore, said signals are used for proliferation control or as process control.
The selection of these reference samples, the addition of further reference samples not listed here, as well as the combination thereof is apparent to the person skilled in the art from the respective application, the experimental objective and the composition of the analytical sample and/or mass spectrometric sample. Alternatively, in another embodiment, stored reference values may be used for the procedure.
Proliferation involves the growth and division of animal cells. The proliferation capability is a measure of the growth and division of animal cells. The proliferation capability is determined in the procedure by means of the degree of coverage or the cell expansion distance.
Migratory capability includes the ability of cells to change location independently. This active independent (self-acting) change of location of animal cells is cell migration. The migration capability preferably includes the undirected spontaneous movement, the directed chemotactic movement and/or the change of the movement speed (chemokinetics).
Cell-specific mass spectrometric signal means that the mass spectrometric signal has been generated by any component of the animal cell. The cell-specific mass spectrometric signal is assignable, preferably uniquely assignable to the animal cells used in the mass spectrometric sample. The components of the animal cell can be proteins, such as ribosomes, peptides, nucleic acids, lipids, and/or other components of the animal cell. Preferably, substances secreted by the animal cell into the environment are not included.
A cell-specific mass spectrometric signature has a selection of one or more cell-specific mass spectrometric signals which, for example, due to the intensity, position/mass range and/or distance thereof from other mass spectrometric signals, are suitable for the unambiguous identification of animal cells at the measured measuring position, even in the presence of nutrient medium, cytotoxic factors, matrix and other components of the mass spectrometric samples. Preferably, the at least one selected cell-specific mass spectrometric signal is distinctive. That is, it is clearly distinguishable from the non-cell-specific mass spectrometric signals and from the background noise by the intensity and mass range (m/z) and/or m/z spacing thereof from other mass spectrometric signals. Cell-specific mass spectrometric signals whose intensity is largely encompassed by the scattering range of the background noise do not count as distinctive cell-specific mass spectrometric signals. Preferably, a spectra library of mass spectrometric signatures is stored in a database. Furthermore, preferably, at least one cell-specific mass spectrometric signature is assigned to each type of animal cell in the spectral library. The stored reference spectra of the cell-specific mass spectrometric signatures may contain, for example, masses, mass tolerances, mean intensities, scattering of intensities and occurrence probabilities. The cell-specific mass spectrometric signatures and/or cell-specific mass spectrometric signals, in particular of the spectrum library, are generated, for example, from multiple measurements on different cell cultivations of the animal cells with and in particular without cytotoxic factors. Preferably, the cell-specific mass spectrometric signals and/or cell-specific mass spectrometric signatures are isolated automatically by computer evaluation.
Preferably, comparisons, matches, assignments, derivations, calculations, determinations, determinations, and other automatable steps of the process are performed by means of a data processing unit. The data processing unit is preferably a computer with corresponding software.

The present invention is explained in more detail below with reference to examples of embodiments shown in the drawings.

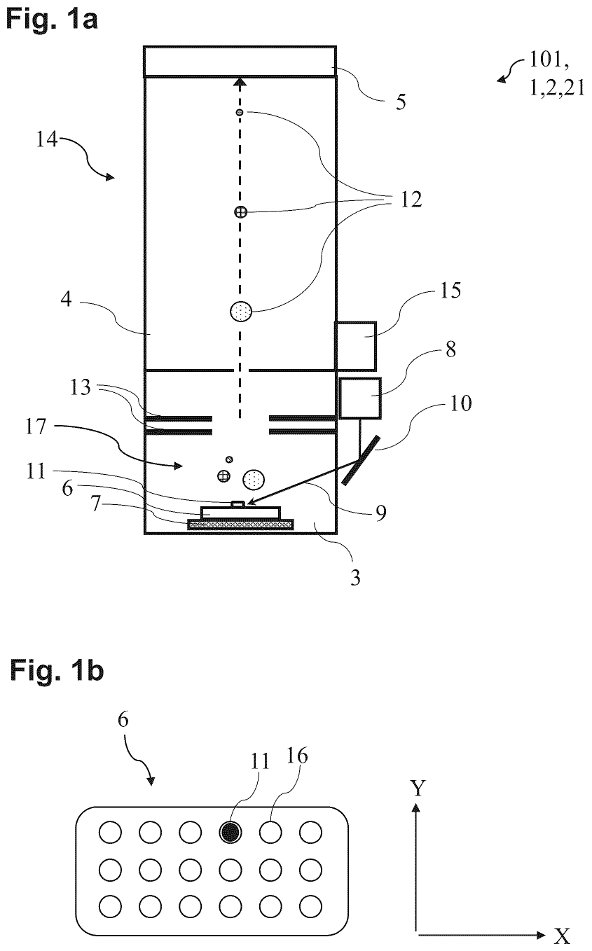

FIG. 1 shows a schematic side view of a spatial resolution mass spectrometer, namely in (a) a schematic view of a simple MALDI-ToF-MS and in (b) a schematic view of a mass spectrometric sample support.

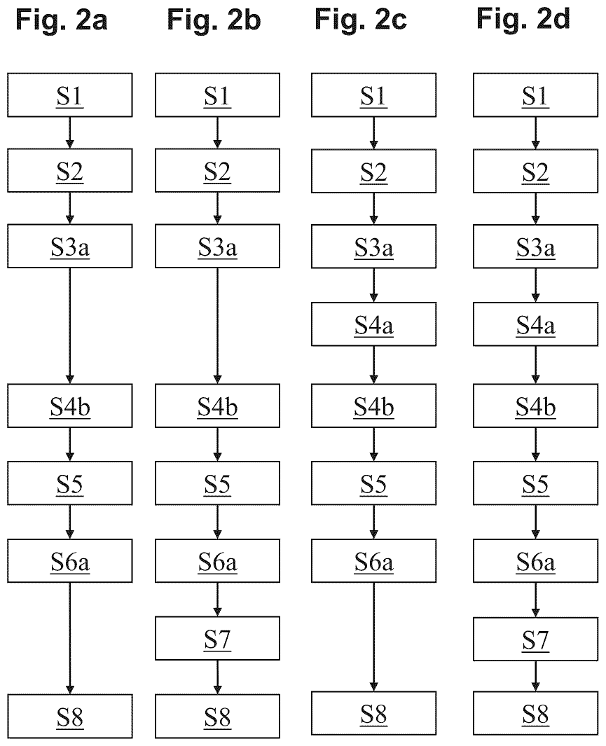

FIG. 2 schematically shows different embodiments of the method according to claim 1, namely in (a) and (b) methods for non-matrix-based spatial resolution mass spectrometers; in (c) and (d) methods for matrix-based spatial resolution mass spectrometers.

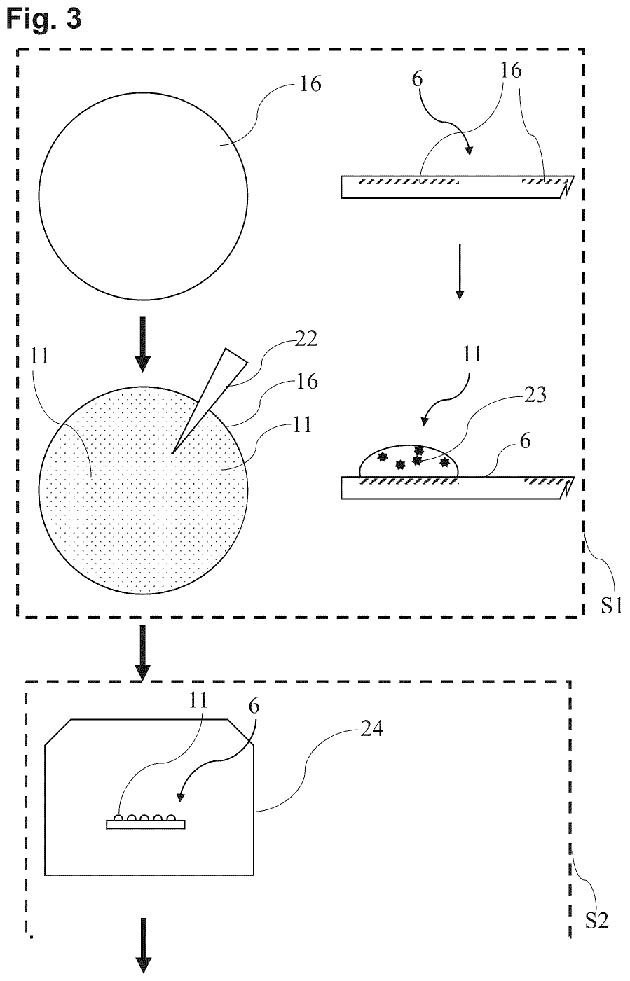

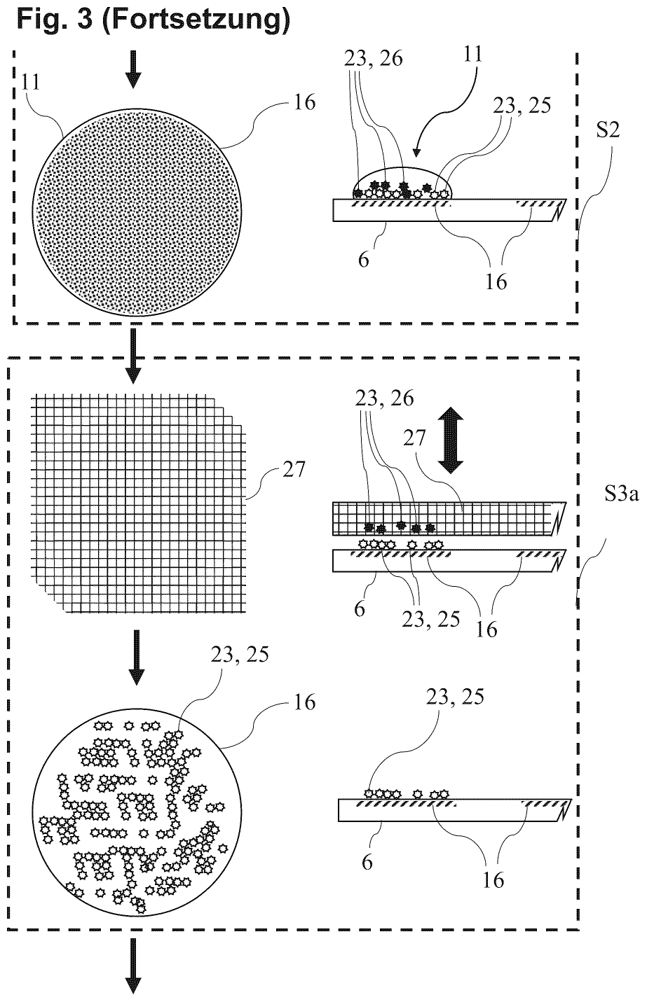

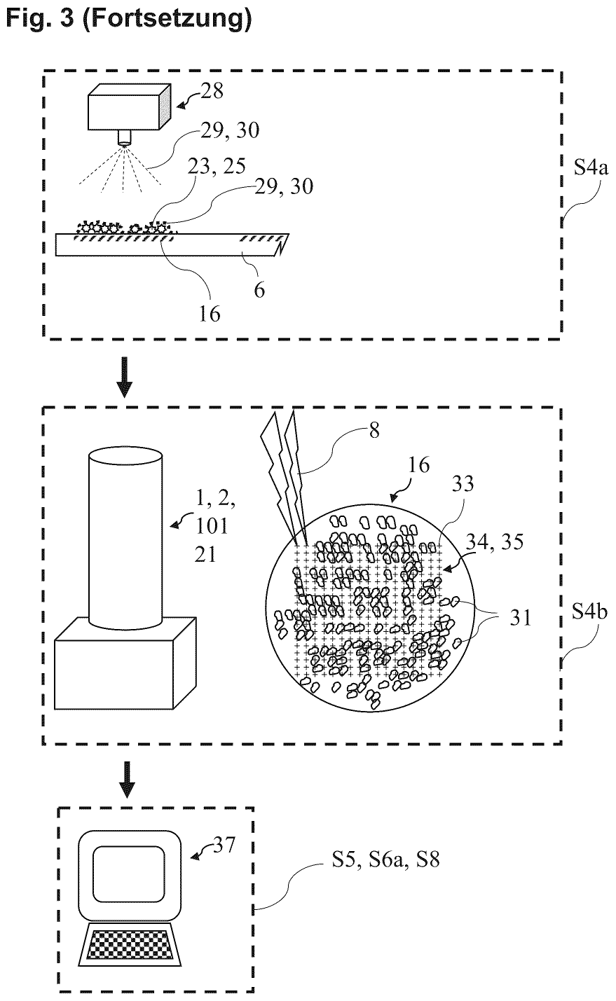

FIG. 3 schematically shows the individual steps of a particularly preferred embodiment of the process according to claim 1 in accordance with the flow chart shown in FIG. 2(c) in detail, with the left-hand side generally showing the top view and the right-hand side showing the associated side view. The example shows an embodiment for a matrix-based spatial resolution mass spectrometer with sample preparation step.

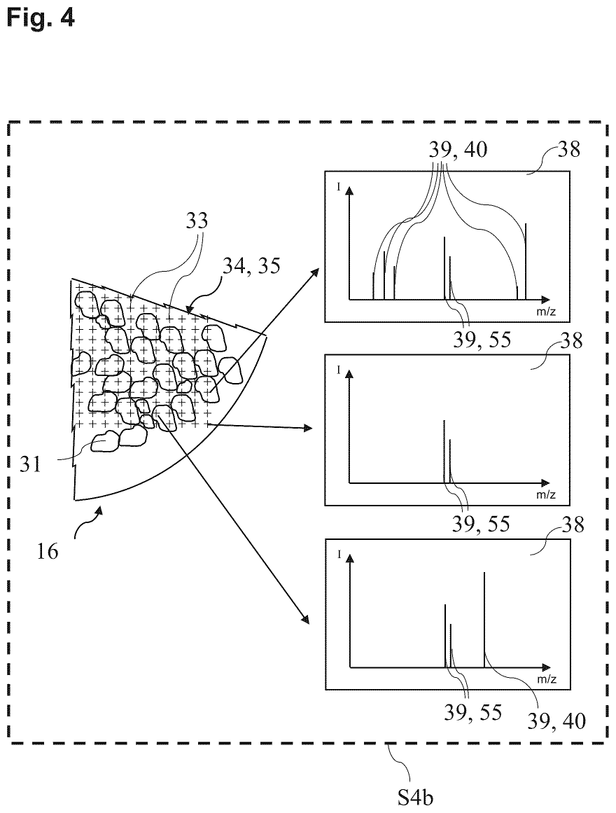

FIG. 4 schematically shows the measuring step A of the method according to claim 1 in detail.

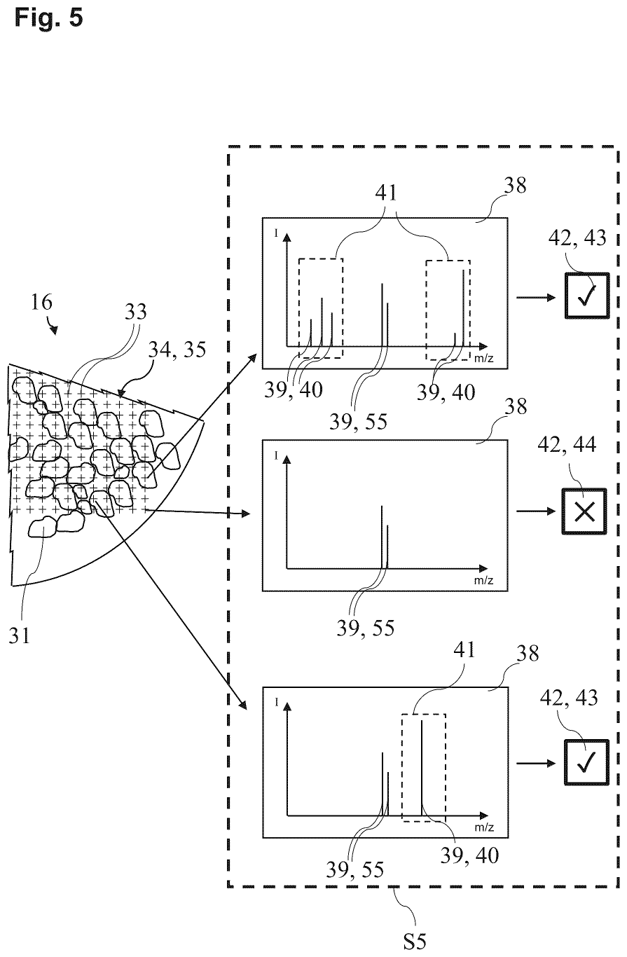

FIG. 5 schematically shows the first evaluation step A according to claim 1 in detail.

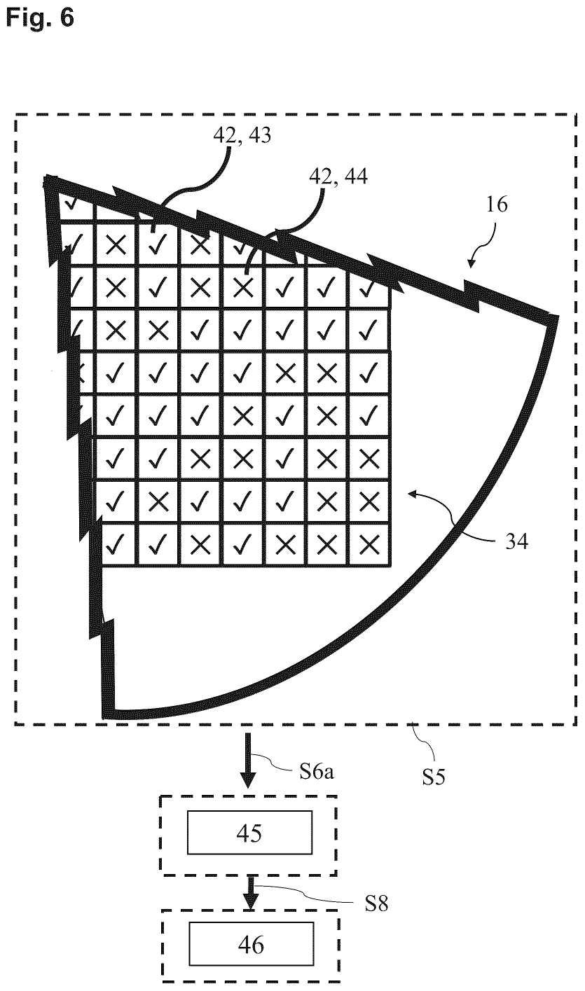

FIG. 6 schematically shows the results of the evaluation steps of the method according to claim 1 corresponding to FIG. 2(c) in detail.

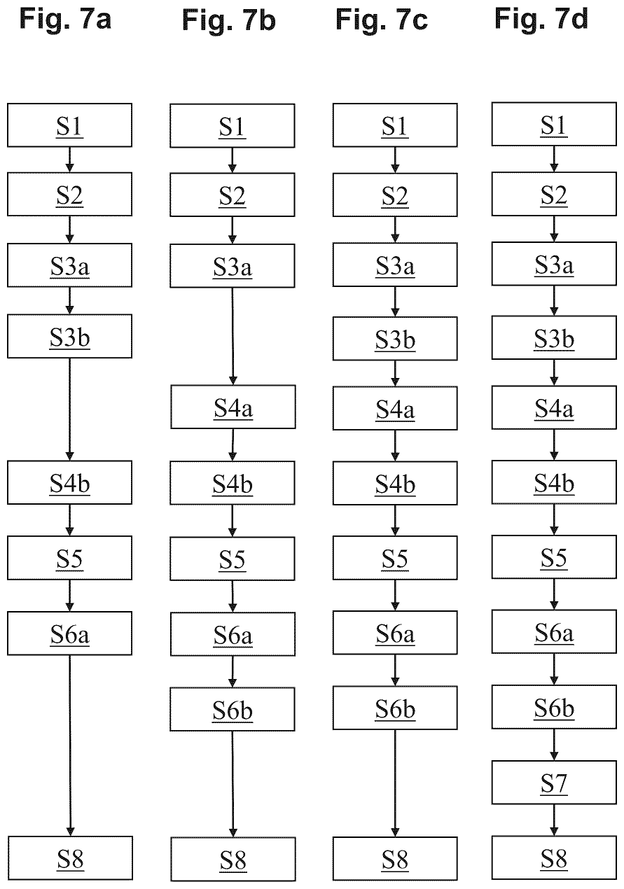

FIG. 7 schematically shows various further preferred embodiments of the method according to claim 1 in combination with selected dependent subclaims, namely in (a) methods for non-matrix-based spatial resolution mass spectrometers; in (b), (c) and (d) methods for matrix-based spatial resolution mass spectrometers. In addition, (b) to (d) have an intermediate evaluation step.

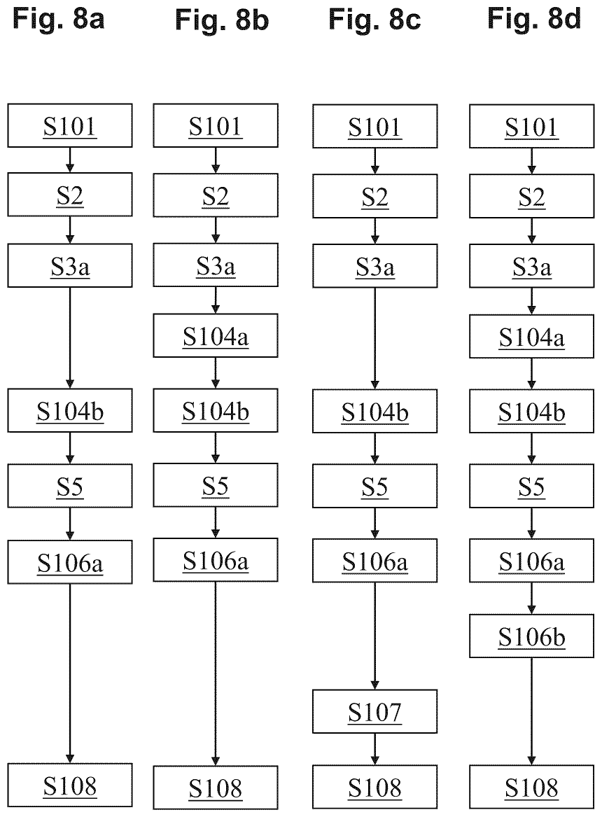

FIG. 8 schematically shows preferred embodiments of the method according to claim 3, namely in (a) and (c) methods for non-matrix-based spatial resolution mass spectrometers; in (b) and (d) methods for matrix-based spatial resolution mass spectrometers.

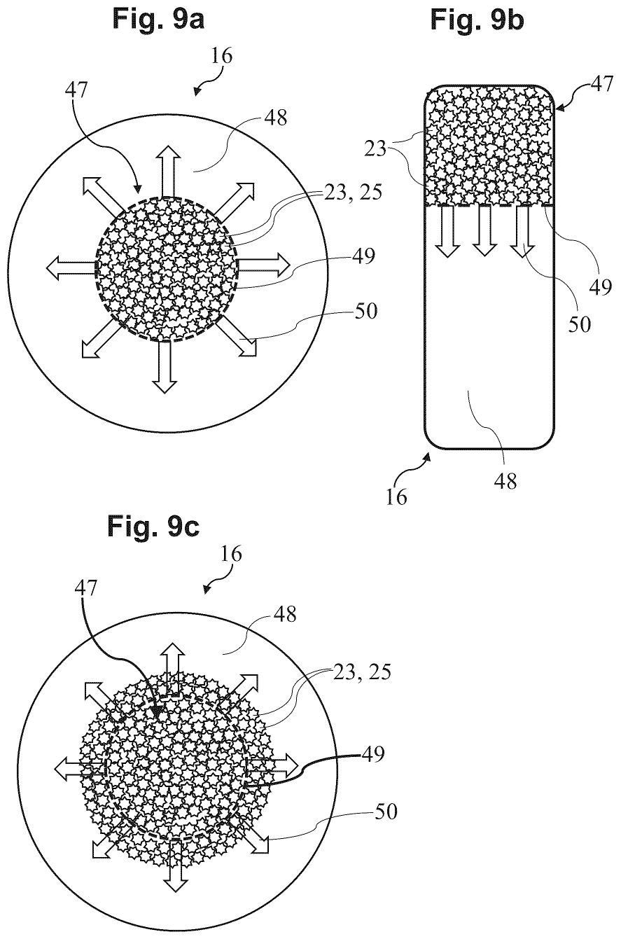

FIG. 9 schematically shows embodiments for the sample spots with the first and the second partial area according to claim 3, namely in (a) a circular sample spot; in (b) an elongated shaped sample spot; in (c) the sample spot from (a) in or after the cultivation step in the absence of or weak cytotoxic effect of the analytical sample.

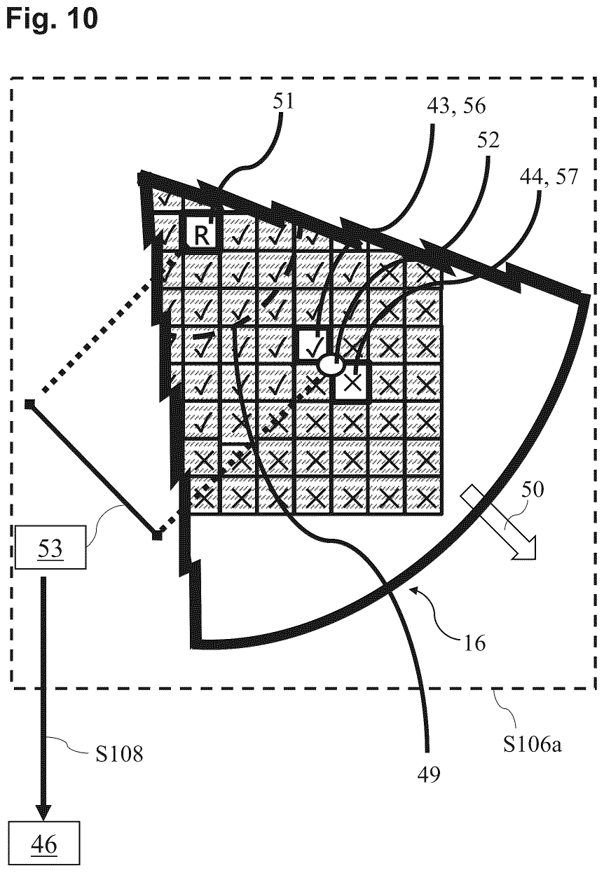

FIG. 10 schematically shows the results of the evaluation steps of the method according to claim 3 corresponding to FIG. 8 (a, b, d) in detail.

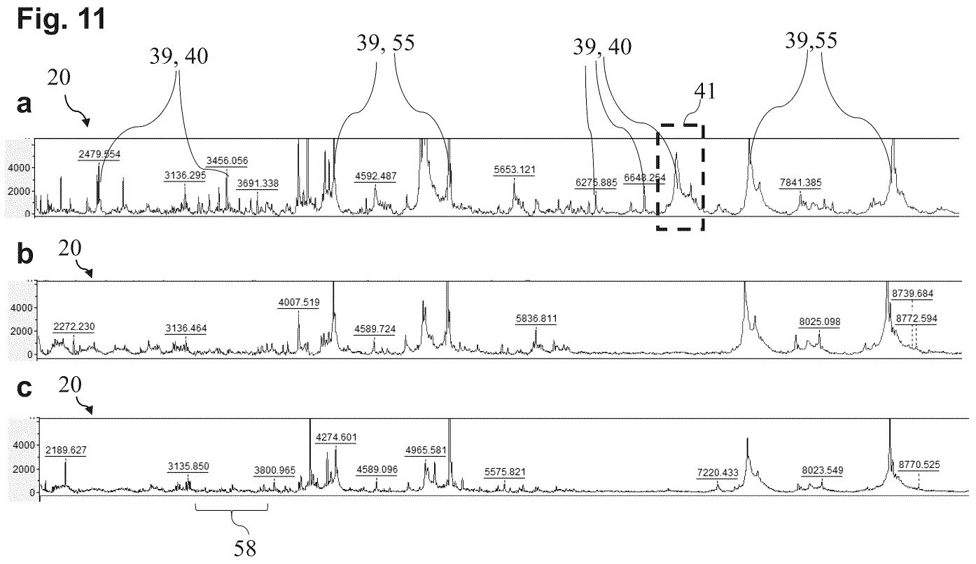

FIG. 11 shows exemplary mass spectra of different reference samples, namely in (a) a first reference sample with a selected type of animal cells and without cytotoxic factor, in (b) a reference sample comprising the same animal cells as in (a) in the presence of a cytotoxic factor, and in (c) a third reference sample, wherein the third reference sample comprises no animal cells and no cytotoxic factor and wherein the third reference sample comprises culture medium and an internal standard. The reference spectra are used here to determine the cell-specific mass spectrometric signals and the cell-specific mass spectrometric signatures.

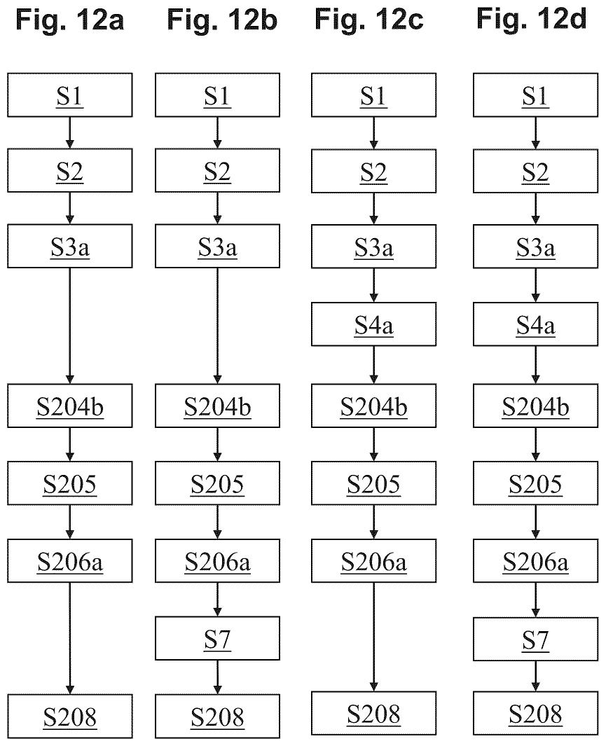

FIG. 12 schematically shows different embodiments of the method according to claim 2, namely in (a) and (b) methods for non-matrix-based mass spectrometers; in (c) and (d) methods for matrix-based mass spectrometers.

FIG. 1a shows a simplified schematic representation of a simple mass spectrometer (MS) 101, more precisely a spatial resolution mass spectrometer 1, more precisely a MALDI-ToF-MS 2, more precisely a matrix-based spatial resolution mass spectrometer 21. This is only an exemplary description of a mass spectrometer. Other system components, such as an ion selector, a fragmentation cell, or an ion reflector, etc., may also be integrated into the system.

The MALDI-ToF-MS 2 comprises an ion source 3 and a time-of-flight mass analyzer 14 (with axial ion injection). The time-of-flight mass analyzer 14 comprises a flight tube 4, a detector 5, acceleration electrodes 13 and an acceleration path 17. The ion source 3 comprises a mass spectrometric sample support 6 positioned on an XY stage 7. The mass spectrometric sample support 6 can be moved in two dimensions X and Y by the XY table 7. Furthermore, the ion source 3 comprises a laser 8 and a mirror and/or lens system 10. The laser 8 generates a laser beam 9. The laser beam 9 is directed via the mirror and/or lens system 10 onto a prepared mass spectrometric sample 11 on the mass spectrometric sample support 6. The laser beam 9 generates sample ions 12 from the prepared mass spectrometric sample 11. These sample ions 12 are accelerated by the accelerating electrodes 13 and pass through the flight tube 4 of the time-of-flight mass analyzer 14. Here the sample ions 12 are separated based on mass and charge. Finally, the sample ions 12 are detected with the detector 5. The vacuum in the flight tube 4 is generated by a vacuum pump 15.

The position of the laser beam 9 on the sample plate 6 is changed by moving the mirror/lens system 10 and/or by moving the XY table 7. Preferably, the laser beam has a beam diameter of 1 nm to 300 μm when it hits the mass spectrometric sample. Preferably, the laser beam has a beam diameter of 100 nm to 100 μm when it hits the mass spectrometric sample. Furthermore, the laser beam preferably has a beam diameter of 1 μm to 10 μm when it hits the mass spectrometric sample.

In the case of a MALDI-ToF-MS 2, the mass spectrometric sample is prepared by applying a matrix 29 to the mass spectrometric sample 11 and then drying it.

The mass spectrometer 101 can operate in a positive or in a negative ion mode. The mass spectrometers 101 are divisible into matrix-based mass spectrometers comprising matrix-based spatial resolution mass spectrometers 21, and non-matrix-based mass spectrometers comprising spatial resolution non-matrix-based mass spectrometers. A matrix-based mass spectrometer requires an additional chemical substance, the matrix 29, to ionize the molecules in the ion source comprising the analytes of the mass spectrometric sample 11 and to generate sample ions 12. In general, this is applied as a liquid. This leads to co-crystallization with the analyte on drying. In this process, the analyte molecules are “incorporated” into the crystals of the matrix 29 as the crystals form. Often small organic molecules are chosen as the main matrix substance, which strongly absorb energy at the laser wavelength used (e.g. nitrogen laser at a wavelength of 337.1 nm). For example, sinapic acid, 2.5-dihydroxybenzoic acid, α-cyanohydroxycinnamic acid, 2,4,6-trihydroxyacetophenone or are used as the matrix main substance. These are usually mixed together with solvents and other substances to form the matrix 29. Water and organic solvents such as acetonitrile or ethanol are often used as solvents. As another substance, trifluoroacetic acid (TFA) is often used as a counter ion source to generate [M+H] ions. An example of a matrix 29 is sinapic acid in acetonitrile:water:TFA (50:50:0.1). Short high-energy laser pulses then lead to explosive particle detachments at the surface of the crystal after relaxation in the crystal lattice. Together with the matrix 29, the enclosed analyte molecules are transferred into the vacuum of the mass spectrometer 101 and thus become accessible for mass spectrometric analysis.

It should be explicitly noted here that alternative mass spectrometers 101 and in particular alternative spatial resolution mass spectrometers 1 with alternative ion source 3 generating ions from mass spectrometric samples on a mass spectrometric sample support 6, such as desorption electrospray ionization (DESI)-MS, can also be used within the scope of the invention.

The mass spectrometers 101 may also include time-of-flight mass analyzers 14 with orthogonal ion injection, electrostatic ion traps (e.g. Orbitrap®), radio frequency ion traps, ion cyclotron resonance ion traps and quadrupole mass filters. Other system components, such as an ion guidance system, a mass filter, ion mobility separators and fragmentation cells, etc., may also be integrated into the mass spectrometric system.

FIG. 1b shows a simplified schematic representation of a top view of a simple mass spectrometric sample support 6. The shift directions of the position of the laser beam 9 on the sample plate 6 in the X-direction and Y-direction by means of the XY-table 7 and/or by means of the mirror and or lens system 10 is also indicated. The mass spectrometric sample support 6 has sample spots 16. Mass spectrometric samples 11 are applied to the sample spots 16. A mass spectrometric sample support 6 has at least one, but usually several sample spots 16. The sample points 16 are preferably designed as circular surfaces. Preferably, the sample spot has a diameter of 0.1 mm to 10 cm. Preferably, the sample spot has a diameter of 0.2 mm to 8 cm. Preferably, the sample spot has a diameter of 0.5 mm to 4 cm. Particularly preferably, the sample spot has a diameter of 1 mm to 1 cm. Very preferably, the sample spot has a diameter of 2 mm to 6 mm. The diameter of the sample spot can be chosen, for example, depending on the beam diameter, the concentration of the animal cells and/or the cell size of the animal cells. In order to promote ionization at least in the area of the sample spots 16, the surface of the mass spectrometric sample support 6 is conductive. Such a mass spectrometric sample support 6 consists, for example, of a polymer such as polypropylene, of ceramic or glass, whereby the mass spectrometric sample support 6 can be coated with a layer of stainless steel. The polymer, ceramic or glass may also contain a conductive material such as carbon black. Alternatively, the mass spectrometric sample support 6 consists of a conductive metal, for example. Mass spectrometric sample supports 6 usually have 48 to 96 sample spots 16. In addition, said supports can have a plurality of reference regions.

FIG. 2 schematically shows various preferred embodiments with the individual steps of the method according to claim 1. The process steps are preferably carried out in the sequential order shown.

Thus FIG. 2a shows a particularly preferred embodiment of the method in which the following steps are carried out: Sample provision step A S1, cultivation step S2, liquid removal step S3a, measurement step A S4b, first evaluation step A S5, second evaluation step A S6a, fourth evaluation step A S8. The cytotoxic effect 46 is derived directly from the degree of coverage 45 in the fourth evaluation step A S8. However, typical intermediate calculations known to the person skilled in the art are not excluded within the scope of the direct derivation. This preferred embodiment of the method is used for non-matrix based spatial resolution mass spectrometers. A true-to-position application of a matrix 29 does not take place.

The process thus has the following features:

A method for determining the cytotoxic effect 46 of an analytical sample on animal cells 23, comprising the following steps:
a sample provision step A S1 in which a mass spectrometric sample 11 comprising animal cells 23, culture medium and the analytical sample potentially having a cytotoxic factor is provided on at least one sample spot 16 of a mass spectrometric sample support 6;
a cultivation step S2 in which the mass spectrometric sample 11 is incubated on the sample spot 16 of the mass spectrometric sample support 6 in a cultivation device 24;
a liquid removal step S3a, in which residual liquid of the mass spectrometric sample 11 is removed from the sample spot 16;
a measurement step A S4b in which spatially resolved mass spectra 38 are recorded at a plurality of measuring positions 33 in at least a partial area 34 of the sample spot 16 by means of a non-matrix-based spatial resolution mass spectrometer 1;
a first evaluation step A S5 in which each spatially resolved mass spectrum 38 is analyzed for the presence of a cell-specific mass spectrometric signature 41 for the animal cells 23 and a cell presence value 42 is assigned to each measuring position 33;
a second evaluation step A S6a, in which a degree of coverage 45 by animal cells 23 is determined for at least the partial area 34 of the sample spot 16 from the cell presence values 42 of the measuring positions 33;
a fourth evaluation step A S8, in which the cytotoxic effect 46 of the analytical sample on the animal cells 23 is derived from the determined degree of coverage 45 of the second evaluation step A S6a.

FIG. 2b shows an alternative preferred embodiment of the method in which the following steps are performed: Sample provision step A S1, cultivation step S2, liquid removal step S3a, measurement step A S4b, first evaluation step A S5, second evaluation step A S6a, third evaluation step A S7, fourth evaluation step A S8. The cytotoxic effect 19 is derived in the fourth evaluation step A S8 from the proliferation capability of the animal cells and thus indirectly from the degree of coverage 45. This preferred embodiment of the method is used for non-matrix based spatial resolution mass spectrometers. A true-to-position application of a matrix 29 does not take place.

The process thus has the following features:

A method for determining the cytotoxic effect 46 of an analytical sample on animal cells 23, comprising the following steps:
a sample provision step A S1 in which a mass spectrometric sample 11 comprising animal cells 23, culture medium and the analytical sample potentially having a cytotoxic factor is provided on at least one sample spot 16 of a mass spectrometric sample support 6;
a cultivation step S2 in which the mass spectrometric sample 11 is incubated on the sample spot 16 of the mass spectrometric sample support 6 in a cultivation device 24;
a liquid removal step S3a, in which residual liquid of the mass spectrometric sample 11 is removed from the sample spot 16;
a measurement step A S4b in which spatially resolved mass spectra 38 are recorded at a plurality of measuring positions 33 in at least a partial area 34 of the sample spot 16 by means of a non-matrix-based spatial resolution mass spectrometer;
a first evaluation step A S5 in which each spatially resolved mass spectrum 38 is analyzed for the presence of a cell-specific mass spectrometric signature 41 for the animal cells 23 and a cell presence value 42 is assigned to each measuring position 33;
a second evaluation step A S6a, in which a degree of coverage 45 by animal cells 23 is determined for at least the partial area 34 of the sample spot 16 from the cell presence values 42 of the measuring positions 33;
a third evaluation step A S7, in which a proliferation capability is derived from the determined degree of coverage 45;
a fourth evaluation step A S8, in which the cytotoxic effect 46 of the analytical sample on the animal cells 23 is indirectly derived from the determined degree of coverage 45 of the second evaluation step A S6a, for which purpose the cytotoxic effect 46 of the analytical sample on the animal cells 23 is derived from the derived proliferative capability of the third evaluation step A S7.

FIG. 2c shows a particularly preferred alternative embodiment of the method in which the following steps are carried out: Sample provision step A S1, cultivation step S2, liquid removal step S3a, sample preparation step A S4a, measurement step A S4b, first evaluation step A S5, second evaluation step A S6a, fourth evaluation step A S8. The cytotoxic effect 19 is derived directly from the degree of coverage 45 in the fourth evaluation step A S8. This preferred embodiment of the method is used for matrix-based spatial resolution mass spectrometers 1, 21. Before measurement step A S4b, a true-to-position application of a matrix 29 takes place in sample preparation step A S4a.

The process thus has the following features:

A method for determining the cytotoxic effect 46 of an analytical sample on animal cells 23, comprising the following steps:
a sample provision step A S1 in which a mass spectrometric sample 11 comprising animal cells 23, culture medium and the analytical sample potentially having a cytotoxic factor is provided on at least one sample spot 16 of a mass spectrometric sample support 6;
a cultivation step S2 in which the mass spectrometric sample 11 is incubated on the sample spot 16 of the mass spectrometric sample support 6 in a cultivation device 24;
a liquid removal step S3a, in which residual liquid of the mass spectrometric sample 11 is removed from the sample spot 16;
a sample preparation step A S4a, in which a preparation of the sample spot 16 is conducted by a true-to-position application of a matrix 29 to at least the one partial area 34 of the sample spot 16;
a measuring step A S4b, in which spatially resolved mass spectra 38 are recorded at a plurality of measuring positions 33 in at least a partial area 34 of the sample spot 16 by means of a matrix-based spatial resolution mass spectrometer 1;
a first evaluation step A S5 in which each spatially resolved mass spectrum 38 is analyzed for the presence of a cell-specific mass spectrometric signature 41 for the animal cells 23 and a cell presence value 42 is assigned to each measuring position 33;
a second evaluation step A S6a, in which a degree of coverage 45 by animal cells 23 is determined for at least the partial area 34 of the sample spot 16 from the cell presence values 42 of the measuring positions 33;
a fourth evaluation step A S8, in which the cytotoxic effect 46 of the analytical sample on the animal cells 23 is derived from the determined degree of coverage 45 of the second evaluation step A S6a.

FIG. 2d shows a preferred alternative embodiment of the method in which the following steps are carried out: Sample provision step A S1, cultivation step S2, liquid removal step S3a, sample preparation step A S4a, measurement step A S4b, first evaluation step A S5, second evaluation step A S6a, third evaluation step A S7, fourth evaluation step A S8. The cytotoxic effect is derived in the fourth evaluation step A S8 from the proliferation capability of the animal cells and thus indirectly from the degree of coverage 45. This preferred embodiment of the method is used for matrix-based spatial resolution mass spectrometers 1, 21. Before measurement step A S4b, a true-to-position application of a matrix 29 takes place in sample preparation step A S4a.

The process thus has the following features:

A method for determining the cytotoxic effect 46 of an analytical sample on animal cells 23, comprising the following steps:
a sample provision step A S1 in which a mass spectrometric sample 11 comprising animal cells 23, culture medium and the analytical sample potentially having a cytotoxic factor is provided on at least one sample spot 16 of a mass spectrometric sample support 6;
a cultivation step S2 in which the mass spectrometric sample 11 is incubated on the sample spot 16 of the mass spectrometric sample support 6 in a cultivation device 24;
a liquid removal step S3a, in which residual liquid of the mass spectrometric sample 11 is removed from the sample spot 16;
a sample preparation step A S4a, in which a preparation of the sample spot 16 is conducted by the true-to-position application of a matrix 29 to at least a partial area 34 of the sample spot 16;
a measuring step A S4b, in which spatially resolved mass spectra 38 are recorded at a plurality of measuring positions 33 in at least a partial area 34 of the sample spot 16 by means of a matrix-based spatial resolution mass spectrometer 1;
a first evaluation step A S5 in which each spatially resolved mass spectrum 38 is analyzed for the presence of a cell-specific mass spectrometric signature 41 for the animal cells 23 and a cell presence value 42 is assigned to each measuring position 33;
a second evaluation step A S6a, in which a degree of coverage 45 by animal cells 23 is determined for at least the partial area 34 of the sample spot 16 from the cell presence values 42 of the measuring positions 33;
a third evaluation step A S7, in which a proliferation capability is derived from the determined degree of coverage 45;
a fourth evaluation step A S8, in which the cytotoxic effect 46 of the analytical sample on the animal cells 23 is indirectly derived from the determined degree of coverage 45 of the second evaluation step A S6a, for which purpose the cytotoxic effect 46 of the analytical sample on the animal cells 23 is derived from the derived proliferative capability of the third evaluation step A S7.

FIG. 3 schematically shows in detail the individual steps of the particularly preferred embodiments of the method according to claim 1 corresponding to the flow chart shown in FIG. 2(c). In this context, certain individual steps can also completely or partially show steps of the claims according to claim 2 and/or 3.

In the sample provision step A S1, a mass spectrometric sample 11 is applied to a sample spot 16 on a mass spectrometric sample support 6 by means of an application device 22. In this example, the application is performed using a pipette. The mass spectrometric sample 11 comprises animal cells 23 which are capable of proliferation.

Furthermore, the mass spectrometric sample 11 comprises an analytical sample and culture medium. For exemplary clarification of the procedure, the analytical sample does not exhibit any cytotoxic effect or it does not contain any cytotoxic factors. In this example, the components of the mass spectrometric sample 11 are already applied in a mixture. In other preferred embodiments, the components of the mass spectrometric sample 11 are partially applied sequentially or separately from each other. Furthermore, the use of pre-prepared mass spectrometric sample supports 6 is also included, which for example already have the animal cells 23 and/or the culture medium in dried form on the sample spots 16. In this example, the components of the mass spectrometric sample 11 are applied in the form of a mixed solution/suspension. In addition to the mass spectrometric sample 11, a reference sample is also applied to another sample spot 16, which comprises the animal cells 23 and the culture medium, but no cytotoxic factor or analytical sample (not shown). The reference sample is treated in the same way as the mass spectrometric sample 11 and later used in the evaluation to generate a reference value.

The animal cells 23 are, for example, proliferation-capable adherent-growing cells of continuous cell lines, such as CHO or MDCK cells.

After application of the mass spectrometric sample 11, the animal cells 23 are cultivated in cultivation step S2. For this purpose, the mass spectrometric sample support 6 is placed in a cultivation device 24. In this example, the cultivation device 24 is a cell culture cabinet. This is where the proliferation of animal cells 23 takes place. If the animal cells 23 have not yet been provided adhered, adherence of the animal cells 23 also occurs here if no cytotoxic factor is present. Likewise, proliferation of animal cells 23 occurs in this step when no cytotoxic factor is present. The cultivation device 24 provides optimal growth conditions, i.e. proliferation conditions, for the animal cells 23. It controls humidity, temperature, CO₂ content, O₂ content and/or other parameters in the cultivation device 24. The duration of the cultivation is set on the reference sample or on the basis of a previously determined time. Thus, with sufficient cultivation time in the cultivation device 24, an increase in the degree of coverage 45 in the reference sample is detectable. After cultivation, adherent animal cells 23, and non-adherent animal cells 23, 26 are present at sample spot 16. The non-adherent animal cells 23, 25 are, for example, animal cells 23 that are still in suspension and have not been able to settle and adhere or dead adherent animal cells 23 that have detached again.

In the following liquid removal step S3a, the liquid components, such as the culture medium and buffer, of the mass spectrometric sample 11 are removed. The adherent animal cells 23,25 remain at their adhered position on the sample spot 16. This is done, for example, by means of a liquid suction device 27 such as with an absorbent material which is brought into contact with the mass spectrometric sample 11 on the sample spot 16. When the liquid of the mass spectrometric sample 11 is sucked up, non-adherent animal cells 26 are also partially or largely completely removed. The result at the end of the liquid removal step S3a is (largely) dry sample spots 16.

Since in this example a matrix-based spatial resolution mass spectrometer 1 is used in measurement step A S4b, the upstream sample preparation step A S4a is carried out before the measurement, which is omitted for non-matrix-based spatial resolution mass spectrometers. The mass spectrometric sample support 6 is sprayed with a matrix 29 by means of a spraying device 28 in sample preparation step A S4a. The spray device 28 generates matrix microdroplets 30 and/or matrix nanodroplets. Furthermore, the spraying device 28 is adjusted and designed in such a way that the already applied matrix droplets can dry during the application of the matrix 29. This is achieved by adjusting the spraying rate of the spraying device, by keeping the matrix droplets as small as possible and/or by drying the surface of the mass spectrometric sample support 6. In addition, a highly volatile matrix 29 can be used. This ensures that the matrix 29 is applied in a true-to-position manner. This ensures that no or only a briefly predominant and thin continuous matrix film forms on the sample spot 16. Among other things, the applied matrix 29 disrupts the animal cells 23, producing disrupted animal cells 31. It is crucial in the case of true-to-position application that the cell components and cell contents of the animal cells 23 remain largely in the position of the respective animal cell 23.

After drying the matrix 29, the mass spectrometric sample 11 is measured by a matrix-based spatial resolution mass spectrometer 21 in measuring step A S4b. In this example, the matrix-based spatial resolution mass spectrometer 21 is a MALDI-ToF-MS 2. For this purpose, spatially resolved mass spectra 38 are recorded at a plurality of measuring positions 33 in a partial area 34 of the sample spot 16. The measuring positions 33 are distributed in the form of a grid 35 over the partial area 34 of the sample spot 16. In this example, the partial area 34 is spanned by the grid 35.

The spatially resolved mass spectra 38 are forwarded to a data processing unit 37 in the form of a computer with software, which processes and evaluates them. The data processing unit 37 performs the evaluation steps. In this example, it performs the first evaluation step A S5, the second evaluation step A S6a and the fourth evaluation step A S8. It then gives a result indicating whether the analytical sample had a cytotoxic effect. In addition, the degree of cytotoxic effect 46 is indicated. The data processing unit 37 is also used to control the matrix-based spatially resolved MS 21 in the measuring step A S4b and to store the generated data.

FIG. 4 schematically shows the measuring step A S4b of the method according to claim 1 in detail. At least one spatially resolved mass spectrum 38 is recorded from each measuring position 33 on the partial area 34 of the sample spot 16. The spatially resolved mass spectrum 38 has mass spectrometric signals 39. These are produced, among other things, by residues of the culture medium, the animal cells 23, and the analytical sample. The contents and components of the animal cells 23 generate cell-specific mass spectrometric signals 40. These are distinguished from non-cell-specific mass spectrometric signals 55 with the aid of a database. The database is preferably generated beforehand using reference samples of the individual components of the mass spectrometric sample 11 or created from other databases. The database includes reference data (reference values) and/or reference data sets. Reference data and reference data sets may include spectrometric measurement data or data derived therefrom. Said data may originate from animal cells 23, culture media, matrices 29, standards, cytotoxic factors (i.e., bacteria, viruses and/or fungi or components of these groups; chemical substances, and other potentially cytotoxic factors), cytotoxicity factor-neutralizing factors, and/or from other components of an analytical sample. Reference data or a reference data set may comprise a mass spectrum and/or a data n-tuple derived from a mass spectrum. An example of a data n-tuple is a list of abundances in the mass spectrometric signal 39 and narrow mass channels associated with them, plus any meta-information about the mass spectrum, if applicable. Derived data may include, for example, peak lists of the most prominent mass spectrometric signals 39 generated from the original mass spectra, or otherwise reduced data, e.g. using baseline subtraction, derivation, noise removal and the like.

In matrix-based spatial resolution mass spectrometers 21, after the sample preparation step A, B S4a, S104a, instead of the largely intact animal cells 23, there are now largely disrupted animal cells 31 at the measuring positions 33. If a disrupted animal cell 31 is present at a measuring position 33, the spatially resolved mass spectrum 38 also shows cell-specific mass spectrometric signals 40. In non-matrix based spatial resolution mass spectrometers, the mass spectrometric signals 40 are largely generated by intact animal cells 23.

Thus, on one part of the measuring positions 33 in FIG. 5 there are disrupted animal cells 31 and on another part of the measuring positions 33 there are no disrupted animal cells 31 or animal cells 23. If no disrupted animal cell 31 is present at a measuring position 33, the spatially resolved mass spectrum 38 does not show cell-specific mass spectrometric signals 40.

FIG. 5 schematically shows the first evaluation step A S5 of the method according to claim 1 in detail. First, each spatially resolved mass spectrum 38 is analyzed for the presence of cell-specific mass spectrometric signatures 41. Said signatures are identified by the data processing unit 37 by means of a comparison of the spatially resolved mass spectra 38 of the individual measuring positions 33 of the mass spectrometric sample 11 comprising the analytical sample or data derived therefrom with created or stored mass spectra of reference samples or data derived therefrom. The matching procedure determines the degree of agreement between the measurement data or data derived therefrom of the mass spectrometric sample comprising the analytical sample and the measurement data or data derived therefrom of the reference samples that have been generated or stored. For example, the degree of match can be determined using similarity measures (“scores”) or logarithms thereof (“log—score—”), as used for example by commercial systems such as the MALDI Biotyper® (Bruker). Furthermore, a classifier, e.g. in the form of a calculation rule, can also be used for classification, having previously been determined by means of classifier training. Other methods are known to the person skilled in the art.

The data processing unit 37 searches for cell-specific mass spectrometric signatures 41 in each spatially resolved mass spectrum 38 to determine whether an animal cell was present at the measurement location 33. Preferably, the same mass spectrometric signature 41 is used for each measuring position 33 of a sample spot 16. Depending on the presence of a cell-specific mass spectrometric signature 41 in the respective spatially resolved mass spectrum 38, a cell presence value 42 is assigned to each measuring position 33. When a cell-specific signature 41 is found in a spatially resolved mass spectrum 38, a positive cell presence value 43 is assigned to measuring position 33, indicating the presence of an animal cell 23 at measuring position 33. If no cell-specific signature 41 is found in a spatially resolved mass spectrum 38, a negative cell presence value 44 is assigned to measuring position 33, indicating the absence of an animal cell 23.

FIG. 6 schematically shows the results of the evaluation steps: second evaluation step A S6a, and fourth evaluation step A S8 of the method according to claim 1 corresponding to FIG. 2(c) in detail. The schematic representation serves primarily for clarification purposes within the scope of this application. The evaluation steps in FIG. 6 are also carried out by the data processing unit 37. After a cell presence value 42 has been assigned to each measuring position 33 in the first evaluation step A S5, a degree of coverage 45 of the partial area 34 of the sample spot 16 with animal cells 23 is determined therefrom in the second evaluation step A S6a. For this purpose, the partial area 34 of the sample spot 16 is divided according to the grid 35. Subsequently, the proportion of the partial area with positive cell presence values 43 to the total partial area 34 of sample spot 16 is calculated. Alternatively, the number of positive cell presence values 43 is set in relation to the number of negative cell presence values 44.

The cytotoxic effect 46 is then derived from the degree of coverage 45 in the fourth (last) evaluation step S8. For this purpose, the degree of coverage 45 is compared with the degree of coverage 45 of the reference sample and/or with stored degrees of coverage 45. This is followed by the derivation of the cytotoxic effect 46. This also determines whether a cytotoxic factor is present in the mass spectrometric sample 11.

For example, the cytotoxic effect 46 of the cytotoxic factor is graded as follows:
i) No cytotoxic effect 46, wherein the coverage 45 of the mass spectrometric sample 11 comprising the analytical sample corresponds to the coverage 45 of a reference sample without cytotoxic factor. The animal cells 23 were able to proliferate unhindered;
ii) strong cytotoxic effect 46, whereby no animal cells 23 or no proliferation of animal cells 23 could be detected on sample spot 16. There was no adherence, growth and/or cell division of animal cells 23. In this case, the degree of coverage 45 of the mass spectrometric sample 11 comprising the analytical sample preferably corresponds to the degree of coverage 45 of a reference sample with cytostatic-acting cytotoxic factor, or is lower, or no animal cells 23 could be detected on the sample spot 16;
iii) medium cytotoxic effect 46, whereby a degree of coverage 45 of less than 50% of the reference sample could be determined;
iv) weak cytotoxic effect 46, whereby a degree of coverage 45 greater than 50% of the reference sample could be detected.

In an alternative preferred embodiment, the proliferation capability is derived from the degree of coverage 45 (not shown in FIG. 6). Here, the degree of coverage 45 is used to derive how well the animal cells 23 can proliferate under the conditions of the mass spectrometric sample 11. The derivation of the cytotoxic effect 46 then takes place alternatively or additionally from the proliferative capability.

FIG. 7 (a-d) schematically shows various preferred embodiments of the method according to claim 1 in combination with selected dependent subclaims. The process steps are preferably carried out in the sequential order shown. Not all possible combinations are shown. The remaining combinations, however, are easily understood by the skilled person.

FIG. 7a shows a preferred embodiment of the method in which the following steps are conducted: Sample provision step A S1, cultivation step S2, liquid removal step S3a, washing step S3b, measuring step A S4b, first evaluation step A S5, second evaluation step A S6a, fourth evaluation step A S8. Skipping the third evaluation step A S7 simplifies the procedure. In washing step S3b, the mass spectrometric sample support 6 is rinsed with a washing solution, for example buffer. The additional washing step S3b after cultivation reduces non-cell-specific mass spectrometric signals 55, background signals and background noise 58, which are generated, for example, by the culture medium, leaked cell contents of damaged animal cells 23 or other components of the mass spectrometric sample 11. Furthermore, non-adherent animal cells 23, which could produce false positive cell presence values 43 and falsify the result, are removed even more reliably by washing. In particular, the washing step S3b is thus recommended if the method contains in the sample provision step S1 the provision of the animal cells 23 by application of animal cells 23 in suspended form and them not being already present on the sample spot 16. The cytotoxic effect 46 is derived from the degree of coverage 45 in the fourth evaluation step A S8. This preferred embodiment of the method is used for non-matrix based spatial resolution mass spectrometers 1. A true-to-position application of a matrix 29 does not take place.

FIG. 7b shows a preferred embodiment of the method in which the following steps are carried out: Sample provision step A S1, cultivation step S2, liquid removal step S3a, sample preparation step A S4a, measurement step A S4b, first evaluation step A S5, second evaluation step A S6a, intermediate evaluation step A S6b, fourth evaluation step A S8. Similarly, the intermediate evaluation step A S6b can be used in methods with non-matrix-based spatial resolution mass spectrometers 21.

This procedure is preferably carried out with matrix-based spatial resolution mass spectrometers 21 with a reference sample. The intermediate evaluation step A S6b is performed if a reference sample is present. Thus, an assessment of the cytotoxic effect 46 can be carried out in a simple and reliable way. The reference sample can also be included in the calculations elsewhere in the procedure. In the intermediate evaluation step A S6b, a comparison of the degree of coverage 45 of sample spot 16 of the reference sample with the degree of coverage 45 of sample spot 16 of the mass spectrometric sample 11 comprising the analytical sample is performed. For this purpose, the coverage 45 of the reference sample is set as a 100% value and coverage 45 of the mass spectrometric sample 11 comprising the analytical sample is put in relation to it. The cytotoxic effect 46 is derived in the fourth evaluation step A S8 from the comparison of the determined degrees of coverage 45 of the mass spectrometric sample and the reference sample. For this purpose, the cytotoxic effect 46 is classified, for example, on the basis of levels in which the gradations of the cytotoxic effect 46 are assigned to value ranges of the degrees of coverage 45. Typical intermediate calculations known to the skilled person are implied here.

FIG. 7c shows a preferred embodiment of the method in which the following steps are carried out: Sample provision step A S1, cultivation step S2, liquid removal step S3a, washing step S3b, measuring step A S4b, first evaluation step A S5, second evaluation step A S6a, intermediate evaluation step A S6b, fourth evaluation step A S8. This procedure is preferably carried out with matrix-based spatial resolution mass spectrometers 21 with a reference sample and with application of the animal cells 23 in suspended form in sample provision step A S1. The use and inclusion of the reference sample in the evaluation easily leads to a precise and reliable assessment of the cytotoxic effect 46. The S3b washing step also increases the reliability and precision of the process.

FIG. 7d shows a preferred embodiment of the method in which the following steps are carried out: Sample provision step A S1, cultivation step S2, liquid removal step S3a, washing step S3b, measuring step A S4b, first evaluation step A S5, second evaluation step A S6a, intermediate evaluation step A S6b, third evaluation step A S7, fourth evaluation step A S8. This method is preferably carried out with matrix-based spatial resolution mass spectrometers 21 with a reference sample and with application of the animal cells 23 in suspended form in sample provision step A S1, if in addition the proliferative capability of the animal cells 23 is to be determined.

FIG. 8 (a-d) schematically shows preferred embodiments of the method according to claim 3. Steps deviating from the method according to claim 1 are marked with deviating reference signs and the letter B. However, the process steps have the same essential features as the process steps already shown in FIG. 3. The process steps are preferably carried out in the sequential order shown.

Thus FIG. 8a shows a particularly preferred embodiment of the method for non-matrix-based spatial resolution mass spectrometers. In this procedure, the following steps are carried out: Sample provision step B S101, cultivation step S2, liquid removal step S3a, measurement step B S104b, first evaluation step A S5, second evaluation step B S106a, fourth evaluation step B S108. A true-to-position application of a matrix 29 does not take place. The cytotoxic effect 46 is derived from the cell expansion distance 53 in the fourth evaluation step B S108. Typical intermediate calculations known to the skilled person are implied here. Detailed descriptions of the process steps can be found in the figure description for FIG. 8b.

The process thus has the following features:

A method for determining the cytotoxic effect 56 of an analytical sample on animal cells 23 comprising the following steps:
a sample provision step B S101 in which a mass spectrometric sample 11 comprising animal cells 23, nutrient medium and the analytical sample potentially having a cytotoxic factor are provided on at least one sample spot 16 of a mass spectrometric sample support 6, wherein the sample spot 16 has a first and a second partial area 47, 48, wherein the animal cells 23 are provided on the first partial area 47 of the sample spot 16 and no animal cells 23 are provided on the second partial area 48 of the sample spot 16 and wherein the remaining mass spectrometric sample 11 is provided on both partial areas 47, 48;
a cultivation step S2 in which the mass spectrometric sample 11 is incubated on the sample spot 16 of the mass spectrometric sample support 6 in a cultivation device 24;
a liquid removal step S3a, in which residual liquids of the mass spectrometric sample 11 on the sample spot 16 are removed;
a measuring step B S104b, in which spatially resolved mass spectra 38 are recorded at least two measuring positions 56, 57 of the sample spot 16 by means of a non-matrix-based spatial resolution mass spectrometer 1, the first measuring position 33, 56 still having animal cells 23 and the second measuring position 33, 57 having no animal cells 23,
whereby both measuring positions 56, 57 are located on one direction of extension 50 starting from a reference point 51;
a first evaluation step A S5 in which each spatially resolved mass spectrum 38 is analyzed for the presence of a cell-specific mass spectrometric signature 41 for the animal cells 23 and a cell presence value 42 is assigned to each measuring position 33, 56, 57;
a second evaluation step B S106a in which a cell expansion distance 53 is determined from at least one distance from a reference point 51 to an transition point 52, wherein each transition point 52 is calculated from at least the first and second measuring positions 56, 57;
a fourth evaluation step B S108, in which the cytotoxic effect 46 of the analytical sample is derived from the cell expansion distance 53 of the second evaluation step B S106a.

FIG. 8b shows another particularly preferred embodiment of the method for matrix-based spatial resolution mass spectrometers 21. In this procedure, the following steps are carried out: Sample provision step B S101, cultivation step S2, liquid removal step S3a, sample preparation step B S104a, measurement step B S104b, first evaluation step A S5, second evaluation step B S106a, fourth evaluation step B S108. The cytotoxic effect 46 is derived from the cell expansion distance 53 in the fourth evaluation step B S108. Typical intermediate calculations known to the skilled person are implied here.

The process thus has the following features:

A method for determining the cytotoxic effect 56 of an analytical sample on animal cells 23 comprising the following steps:
a sample provision step B S101 in which a mass spectrometric sample 11 comprising animal cells 23, nutrient medium and the analytical sample potentially having a cytotoxic factor is provided on at least one sample spot 16 of a mass spectrometric sample support 6, wherein the sample spot 16 has a first and a second partial area 47, 48, wherein the animal cells 23 are provided on the first partial area 47 of the sample spot 16 and no animal cells 23 are provided on the second partial area 48 of the sample spot 16 and wherein the remaining mass spectrometric sample 11 is provided on both partial areas 47, 48;
a cultivation step S2 in which the mass spectrometric sample 11 is incubated on the sample spot 16 of the mass spectrometric sample support 6 in a cultivation device 24;
a liquid removal step S3a, in which residual liquids of the mass spectrometric sample 11 on the sample spot 16 are removed;
Sample preparation step B S104a, in which a preparation of the sample spot 16 is conducted by the true-to-position application of a matrix 29 to at least a part of both partial areas 47,48 of the sample spot 48;
a measuring step B S104b, in which spatially resolved mass spectra 38 are recorded at least two measuring positions 56, 57 of the sample spot 16 by means of a matrix-based spatial resolution mass spectrometer 1, the first measuring position 33, 56 still having animal cells 23 and the second measuring position 33, 57 having no animal cells 23,
whereby both measuring positions 56, 57 are located on one direction of extension 50 starting from a reference point 51;
a first evaluation step A S5 in which each spatially resolved mass spectrum 38 is analyzed for the presence of a cell-specific mass spectrometric signature 41 for the animal cells 23 and a cell presence value 42 is assigned to each measuring position 33, 56, 57;
a second evaluation step B S106a in which a cell expansion distance 53 is determined from at least one distance from a reference point 51 to an transition point 52, wherein each transition point 52 is calculated from at least the first and second measuring positions 56, 57;
a fourth evaluation step B S108, in which the cytotoxic effect 46 of the analytical sample is derived from the cell expansion distance 53 of the second evaluation step B S106a.

Exemplary of the other preferred embodiments according to claim 3 or claim 20 (e.g. in FIG. 8 a-d), this embodiment is described in detail herein:
Sample provision step B S101: The animal cells 23 are provided on the first partial area 47. For this purpose, the animal cells 23 with nutrient medium are placed on the first partial area 47 and incubated there. After the animal cells 23 have grown and adhered to the first partial area 47, the residual fluid is removed. In the process, non-adherent animal cells 23 are also removed. Then the remaining mass spectrometric sample 11 without animal cells 23 is added. The remaining mass spectrometric sample 11 includes, among other things, the analytical sample, for example a urine sample potentially containing a cytotoxic factor, and culture medium. The remaining mass spectrometric sample 11 is applied to the entire sample spot 16, i.e. the first partial area 47 and the second partial area 48.
Cultivation step S2: The animal cells 23 are cultivated analogously to the cultivation step S2 in the figure description of FIG. 3.
Liquid removal step S3a: The residual liquids of the mass spectrometric sample 11 on sample spot 16 are removed. This step is analogous to the liquid removal step S3a in the figure description of FIG. 3.
Sample preparation step B S104a: The application is analogous to the sample preparation step A 4a in the figure description of FIG. 3. In this example, the matrix 29 is applied to the entire sample spot 16 in a true-to-position manner.
Measuring step B S104b: Spatially resolved mass spectra 38 are recorded at a plurality of measuring positions 33. In an alternative preferred embodiment, spatially resolved mass spectra 38 are recorded only at a plurality of measuring positions 33 along directions of extension 50 at which the subsequent determination of cell expansion distance 53 takes place. This saves unnecessary measurements.
First evaluation step S5: Each spatially resolved mass spectrum 38 is analyzed for the presence of a cell-specific mass spectrometric signature 41 for the animal cells 23 and a cell presence value 42 is assigned to each measuring position 33. This first evaluation step S5 is carried out analogously to the figure descriptions of FIGS. 4 and 5.
Second evaluation step B S106a: The transition point 52 along a direction of extension is determined. The distance from a reference value to the transition point 52 gives the cell expansion distance. Further details on the second evaluation step B S106a can be found in the explanations for FIG. 10.
Fourth evaluation step B S108: The cytotoxic effect is derived from the cell expansion distance 53 by comparing the cell expansion distance 53 with reference values. These are already available as stored data or are determined with the help of reference samples. Based on the reference values, the cell expansion distance 53 can be assigned to a cytotoxic effect 46. Subsequently, it is output whether the analytical sample had a cytotoxic effect. In addition, it is output how strong the cytotoxic effect was. Preferably, in the fourth evaluation step B S108, the proliferation ability and/or the migration ability of the animal cells and/or the virulence of the analytical sample is directly derived from the cell expansion distance 53.

FIG. 8c shows an alternative preferred embodiment of the method for non-matrix based spatial resolution mass spectrometers. The following steps are conducted in this procedure: Sample provision step B S101, cultivation step S2, liquid removal step S3a, measurement step B S104b, first evaluation step A S5, second evaluation step B S106a, third evaluation step B S107, fourth evaluation step B S108. A true-to-position application of a matrix 29 does not take place. The cytotoxic effect 46 is derived from the proliferation ability and/or migration ability in the fourth evaluation step B S108. Typical intermediate calculations known to the skilled person are implied here.

The process thus has the following features:

A method for determining the cytotoxic effect 56 of an analytical sample on animal cells 23 comprising the following steps:
a sample provision step B S101 in which a mass spectrometric sample 11 comprising animal cells 23, nutrient medium and the analytical sample potentially having a cytotoxic factor is provided on at least one sample spot 16 of a mass spectrometric sample support 6, wherein the sample spot 16 has a first and a second partial area 47, 48, wherein the animal cells 23 are provided on the first partial area 47 of the sample spot 16 and no animal cells 23 are provided on the second partial area 48 of the sample spot 16 and wherein the remaining mass spectrometric sample 11 is provided on both partial areas 47, 48;
a cultivation step S2 in which the mass spectrometric sample 11 is incubated on the sample spot 16 of the mass spectrometric sample support 6 in a cultivation device 24;
a liquid removal step S3a, in which residual liquids of the mass spectrometric sample 11 on the sample spot 16 are removed;
a measuring step B S104b, in which spatially resolved mass spectra 38 are recorded at least two measuring positions 56, 57 of the sample spot 16 by means of a non-matrix-based spatial resolution mass spectrometer 1, the first measuring position 33, 56 still having animal cells 23 and the second measuring position 33, 57 having no animal cells 23,
whereby both measuring positions 56, 57 are located on one direction of extension 50 starting from a reference point 51;
a first evaluation step A S5 in which each spatially resolved mass spectrum 38 is analyzed for the presence of a cell-specific mass spectrometric signature 41 for the animal cells 23 and a cell presence value 42 is assigned to each measuring position 33, 56, 57;
a second evaluation step B S106a in which a cell expansion distance 53 is determined from at least one distance from a reference point 51 to an transition point 52, wherein each transition point 52 is calculated from at least the first and second measuring positions 56, 57;
a third evaluation step B S107, in which a proliferation capability and/or a migration capability of the animal cells 23 is derived from the determined cell expansion distance 53;
a fourth evaluation step B S108, in which the cytotoxic effect 46 of the analytical sample is derived from the proliferation ability and/or the migration ability of the third evaluation step B S107.

FIG. 8d shows an alternative preferred embodiment of the method for matrix-based spatial resolution mass spectrometers 21. The following steps are carried out in this procedure: Sample provision step B S101, cultivation step S2, liquid removal step S3a, sample preparation step B S104a, measurement step B S104b, first evaluation step A S5, second evaluation step B S106a, intermediate evaluation step B S106b, fourth evaluation step B S108. In the intermediate evaluation step B S106b, a comparison of the cell expansion distance 53 of at least one reference sample with the cell expansion distance 53 of the mass spectrometric sample 11 is carried out. For this purpose, the cell expansion distance 53 of the reference sample is set as a 100% value and the cell expansion distance 53 of the mass spectrometric sample 11 is put in relation to it. The cytotoxic effect 46 is derived in the fourth evaluation step B S108 from the comparison of the determined cell expansion distances 53 of the mass spectrometric sample 11 and the reference sample. For this purpose, the cytotoxic effect is classified using levels in which the gradations of the cytotoxic effect are assigned to value ranges of the cell expansion distance 53. Typical intermediate calculations known to the skilled person are implied here.

The process thus has the following features:

A method for determining the cytotoxic effect 56 of an analytical sample on animal cells 23 comprising the following steps:
a sample provision step B S101 in which a mass spectrometric sample 11 comprising animal cells 23, nutrient medium and the analytical sample potentially having a cytotoxic factor is provided on at least one sample spot 16 of a mass spectrometric sample support 6, wherein the sample spot 16 has a first and a second partial area 47, 48, wherein the animal cells 23 are provided on the first partial area 47 of the sample spot 16 and no animal cells 23 are provided on the second partial area 48 of the sample spot 16 and wherein the remaining mass spectrometric sample 11 is provided on both partial areas 47, 48;
a cultivation step S2 in which the mass spectrometric sample 11 is incubated on the sample spot 16 of the mass spectrometric sample support 6 in a cultivation device 24;
a liquid removal step S3a, in which residual liquids of the mass spectrometric sample 11 on the sample spot 16 are removed;
Sample preparation step B S104a, in which a preparation of the sample spot 16 is conducted by the true-to-position application of a matrix 29 to at least a part of both partial areas 47,48 of the sample spot 16;
a measuring step B S104b, in which spatially resolved mass spectra 38 are recorded at least two measuring positions 56, 57 of the sample spot 16 by means of a matrix-based spatial resolution mass spectrometer 1, the first measuring position 33, 56 still having animal cells 23 and the second measuring position 33, 57 having no animal cells 23,
whereby both measuring positions 56, 57 are located on one direction of extension 50 starting from a reference point 51;
a first evaluation step A S5 in which each spatially resolved mass spectrum 38 is analyzed for the presence of a cell-specific mass spectrometric signature 41 for the animal cells 23 and a cell presence value 42 is assigned to each measuring position 33, 56, 57;
a second evaluation step B S106a in which a cell expansion distance 53 is determined from at least one distance from a reference point 51 to an transition point 52, wherein each transition point 52 is calculated from at least the first and second measuring positions 56, 57;
an intermediate evaluation step B S106b, in which a comparison of the cell expansion distance 53 of at least one reference sample with the cell expansion distance 53 of the mass spectrometric sample 11 comprising the analytical sample is carried out and is included in the derivation of the cytotoxic effect 46 in the fourth evaluation step B S108;
a fourth evaluation step B S108, in which the cytotoxic effect 46 of the analytical sample is derived from the cell expansion distance 53 of the analytical sample of the second evaluation step B S106a, including the comparison from the intermediate evaluation step B S106b.

FIG. 9 schematically shows preferred embodiments of the sample spots 16 according to claim 3. For example, FIG. 9a shows a circular sample spot 16 with a first partial area 47 and a second partial area 48 in sample provision step B S101. The animal cells 23 are already provided on the first partial area 47. For clarification purposes, the first partial area 47 is delimited from the second partial area 48 by an imaginary interrupted boundary line 49. The second partial area 48 is free of animal cells 23. The extension of the animal cells 23 onto the second partial area 48 in the following cultivation step S2 preferably takes place in the directions of extension 50. The directions of extension 50 preferably extend orthogonally to the boundary line 49.

FIG. 9b shows an alternative embodiment of the sample spots 16 according to claim 3 in sample provision step B S101. The sample spot 16 is elongated. It also has a first partial area 47 and a second partial area 48. For clarification purposes, the first partial area 47 is delimited from the second partial area 48 by an imaginary interrupted boundary line 49. The animal cells 23 are already provided on the first partial area 47. Here, the cell expansion distance 53 is determined in the later second evaluation step B S106a in only one direction of extension 50 at different positions of the boundary line 49 orthogonal to the boundary line 49.

Further embodiments of the sample spots 16 are also encompassed within the scope of the invention. Thus, further forms of sample spot 16 are included. Also included is the embodiment in which the sample spot 16 is circular and the first partial area 47 and the second partial area 48 are formed such that the adjacent sides of the two partial area 47, 48 form an imaginary boundary line 49 which is straight.

FIG. 9c schematically shows the sample spot 16 from FIG. 9a after cultivation step S2, in which proliferation took place. The animal cells 23 have spread from the first partial area 47 beyond the boundary line 49 to the second partial area 48. The cell expansion distance 53 in the later second evaluation step B S106a is determined in the direction of extension 50.

FIG. 10 schematically shows the second evaluation step B S106a and the fourth evaluation step B S108 of the method according to claim 3 as illustrated in detail, for example, according to FIG. 8 (a-d) and according to FIG. 9c. In particular, the calculation of a transition point 52 and the determination of a cell expansion distance 53 are shown.

In this example, the directions of extension 50 start at the same reference point 51. In this example, the reference point 51 is in the center of the first partial area 47. To determine an transition point 52 on one direction of extension 50, the cell presence values 42 are checked starting from a reference point 51. For this purpose, a change from a positive cell presence value 43 to a negative cell presence value 44 is determined along the direction of extension 50 at two measuring positions 33, 56, 57 adjacent in the direction of extension 50. Thus, in the direction of extension 50 from the reference point 51, there are animal cells 23 in front of the transition point 52 at largely every measuring position 33 and largely no animal cells 23 behind the transition point 52. The transition point 52 is thus calculated in the direction of extension 50 from a first and a second measuring position 56, 57, each with different cell presence values 42, a positive cell presence value 43 and a negative cell presence value 44. The transition point 52 is preferably located centrally between the two measuring positions 56, 57. The distance from the reference point 51 to the transition point 52 gives the cell expansion distance 53. This is preferably determined for several directions of extension 50. This is followed by an averaging of the calculated cell expansion distances 53 of a sample spot 16. The cytotoxic effect 46 of the analytical sample 16 is derived from the averaged cell expansion distance 53.

For example, the cytotoxic effect 46 of the analytical sample is graded as follows:
(i) No cytotoxic effect 46, wherein the cell expansion distance 53 of the mass spectrometric sample 11 comprising the analytical sample corresponds to the cell expansion distance 53 of a reference sample without cytotoxic factor. The animal cells 23 were able to grow and divide unhindered;
(ii) strong cytotoxic effect 56, with no animal cells 23 detected on sample spot 16 or no animal cells 23 detected on the second partial area 48 of sample spot 16. There was no growth or cell division of animal cells 23. The cell expansion distance 53 is largely 0. Alternatively, the animal cells 23 have detached from the first partial area 47, which is why no first measuring position 56 can be detected at the original boundary of the first partial area 47;
(iii) Medium cytotoxic effect 46, with a cell expansion 53 less than 50% of the reference sample;
(iv) weak cytotoxic effect 46, with a cell expansion 53 greater than 50% of the reference sample.

FIG. 11 shows exemplary mass spectra 20 of different mass spectrometric samples 11 with cell-specific mass spectrometric signals 40, non-cell-specific mass spectrometric signals 55 and cell-specific mass spectrometric signatures 41. The mass spectrometer 101 used was a non-spatial-resolution MALDI-ToF. Also, the matrix 29 was not true-to-position applied. Thus, the mass spectra 20 represent sum spectra of an entire sample spot 16 and have not been generated according to the invention. In addition, the mass spectra 20 are not spatially resolved. It is intended here, among other things, to clarify the cell-specific mass spectrometric signals 40, the cell-specific mass spectrometric signatures 41, the non-cell-specific mass spectrometric signals 55 and a possible determination of reference values. In particular, the mass spectrum 20 in FIG. 11 (b) is only an approximate example here, since a small residual number of animal cells 23, which were already present before the cultivation step S2 (cell seed), can generate largely weak cell-specific mass spectrometric signals 40. FIG. 11 (a) shows a mass spectrum 20 of a first reference sample comprising animal cells 23 and culture medium. The animal cells 23 are cells of the continuous human cell line A549. The complex culture medium DMEN with 10% fetal calf serum (FCS) was used as culture medium. The cultivation of the animal cells 23 was carried out for 24 h. A cytotoxic factor was not present. The mass spectrum 20 comprises mass spectrometric signals 39 generated by the animal cells 23, the culture medium, the cytotoxic factor and the matrix 29. It comprises cell-specific mass spectrometric signals 40, non-cell-specific mass spectrometric signals 55 and a selected cell-specific mass spectrometric signature 41.

FIG. 11 (b) shows a mass spectrum 20 of a second reference sample (respectively a mass spectrometric sample comprising an analytical sample with a cytotoxic factor) comprising animal cells 23, culture medium and a cytotoxic factor. The animal cells 23 are also cells of the human continuous cell line A549. The complex culture medium DMEN with 10% fetal calf serum (FCS) was also used as culture medium. The cytotoxic factor is tetradecyltrimethylammonium chloride. The cultivation of the animal cells 23 was conducted for 24 h. The mass spectrum 20 comprises non-cell-specific mass spectrometric signals 55, which were essentially generated by the culture medium, the cytotoxic factor and the matrix 29. Due to the presence of a cytotoxic factor during cultivation step S2, no proliferation of the animal cells 23 occurred. Distinctive cell-specific mass spectrometric signals 40 are not present. Since this example is a sum mass spectrum of the entire sample spot 16 not according to the invention, sporadic weak cell-specific mass spectrometric signals 40 may be detected in this example, which are largely absent when using a spatial resolution mass spectrometer 1. These weak cell-specific mass spectrometric signals 40 are attributable to the small number of animal cells 23 provided at the beginning (cell seed).

Furthermore, weak and low in number mass spectrometric signals 39 can be generated by proteins secreted by animal cells 23, by cell components of destroyed animal cells 23 or by metabolic products which are partially distributed over individual measuring positions 33 of a sample spot 16 during the cultivation step S2. However, these mass spectrometric signals 39 are usually also of low intensity and can, if necessary, be removed to a large extent in a simple manner by an optional washing step S3B prior to spatially dispersed application of the matrix 29. Preferably, however, a mass spectrometric signature 41 is chosen which is largely unaffected by such interferences. This mass spectrometric signature 41 can be determined, for example, with corresponding reference samples. It can also be done, for example, using a summed mass spectrum of the entire sample spot 16 of a non-spatial-resolution MALDI-ToF as in FIG. 11 (b). This data may have been previously stored in a database of the data processing unit 37 and made available to the method.

FIG. 11 (c) shows a mass spectrum 20 of a third reference sample comprising culture medium and an internal standard, wherein the third reference sample does not comprise animal cells 23 and cytotoxic factor. The culture medium and matrix 29 correspond to those in FIGS. 11 (a) and (b). Cell-specific mass spectrometric signals 40 are not present.

From the comparison of the mass spectra 20 of the first reference sample (a) with the second and third reference samples in (b) and (c), the mass spectrometric signals 39 can be assigned to the animal cells 23, the culture medium and the matrix 29. The selected cell-specific mass spectrometric signature 41 in FIG. 11 (a) has cell-specific mass spectrometric signals 40. These reported cell-specific mass spectrometric signals 40 are distinctive. Thus, the cell-specific mass spectrometric signals 40 of the cell-specific mass spectrometric signature 41 have in particular a high intensity difference compared to a plurality of non-cell-specific mass spectrometric signals 55, the background noise 58 and to the intensities in this mass range of the second reference sample (b) and the third reference sample (c).

In particular, the comparison of mass spectra 20 of reference samples in whole or in part as described herein is a preferred option to determine the cell-specific mass spectrometric signals 40 and the cell-specific mass spectrometric signatures 41. Further comparisons with other reference samples are explicitly included. In particular, the determination of cell-specific mass spectrometric signals 40 and of cell-specific mass spectrometric signatures 41 can be performed by means of matching mass spectra 20 stored in a database, in particular of reference samples, and/or data derived therefrom. Further methods for determining cell-specific mass spectrometric signals 40, in particular distinctive cell-specific mass spectrometric signals 40, are sufficiently known to the person skilled in the art. For example, the process may include feature detection, feature selection and/or classifier training. For example, univariate data analysis techniques and/or multivariate data analysis techniques can be used for classifier training. Univariate data analysis procedures include, for example, receiver operator characteristic, t-test, Kruskal-Wallis test and/or ANOVA. Multivariate data analysis techniques include, for example, linear discriminant analysis (LDA), random forest methods, decision trees, principal component analysis (PCA), hierarchical and other clustering methods, pattern recognition, neural networks and/or SVM.

FIG. 12 schematically shows various preferred embodiments with the individual steps of the method according to claim 2. Steps deviating from the method according to claim 1 and claim 3 are marked with deviating reference signs and the letter B. However, the process steps have the same essential features as the process steps already shown in FIG. 3. Thus, various details of the individual steps can be taken from the examples of the method according to claim 1, despite the different identifier (A, B, C). The process steps are preferably carried out in the sequential order shown.

Thus, FIG. 12a shows a particularly preferred embodiment of the method in which the following steps are carried out: Sample provision step A S1, cultivation step S2, liquid removal step S3a, measurement step C S204b, first evaluation step C S205, second evaluation step C S206a, fourth evaluation step C S208. The cytotoxic effect 46 is derived directly from the degree of coverage 45 in the fourth evaluation step C S208. However, typical intermediate calculations known to the person skilled in the art are not excluded within the scope of the direct derivation. This preferred embodiment of the method is used for non-matrix based mass spectrometers. A true-to-position application of a matrix 29 does not take place. The process thus has the following features:

A method for determining the cytotoxic effect 46 of an analytical sample on animal cells 23, comprising the following steps:
a sample provision step A S1 in which a mass spectrometric sample 11 comprising animal cells 23, culture medium and the analytical sample potentially having a cytotoxic factor is provided on at least one sample spot 16 of a mass spectrometric sample support 6;
a cultivation step S2 in which the mass spectrometric sample 11 is incubated on the sample spot 16 of the mass spectrometric sample support 6 in a cultivation device 24;
a liquid removal step S3a, in which residual liquid of the mass spectrometric sample 11 is removed from the sample spot 16;
a measuring step C S204b, in which mass spectra 38 are recorded at a plurality of measuring positions 33 distributed over at least a partial area 34 of the sample spot 16 by means of a non-matrix-based mass spectrometer;
a first evaluation step C S205 in which each mass spectrum 20 is analyzed for the presence of a cell-specific mass spectrometric signature 41 for the animal cells 23;
a second evaluation step C S206a, in which a degree of coverage 45 by animal cells 23 is determined for at least the partial area 34 of the sample spot 16 from the result of the first evaluation step C S205c;
a fourth evaluation step C S208, in which the cytotoxic effect 46 of the analytical sample on the animal cells 23 is derived directly from the determined degree of coverage 45 of the second evaluation step C S206a.

FIG. 12b shows an alternative preferred embodiment of the method in which the following steps are performed: Sample provision step A S1, cultivation step S2, liquid removal step S3a, measurement step C S204b, first evaluation step C S205, second evaluation step C S206a, third evaluation step A S7, in which a proliferation capability is derived from the determined degree of coverage 45, fourth evaluation step CS208. The cytotoxic effect 19 is derived in the fourth evaluation step C S208 from the proliferation capability of the animal cells and thus indirectly from the degree of coverage 45. This preferred embodiment of the method is used for non-matrix based mass spectrometers. A true-to-position application of a matrix 29 does not take place.

The process thus has the following features:

A method for determining the cytotoxic effect 46 of an analytical sample on animal cells 23, comprising the following steps:
a sample provision step A S1 in which a mass spectrometric sample 11 comprising animal cells 23, culture medium and the analytical sample potentially having a cytotoxic factor is provided on at least one sample spot 16 of a mass spectrometric sample support 6;
a cultivation step S2 in which the mass spectrometric sample 11 is incubated on the sample spot 16 of the mass spectrometric sample support 6 in a cultivation device 24;
a liquid removal step S3a, in which residual liquid of the mass spectrometric sample 11 is removed from the sample spot 16;
a measuring step C S204b, in which mass spectra 20 are recorded at a plurality of measuring positions 33 distributed over at least a partial area 34 of the sample spot 16 by means of a non-matrix-based mass spectrometer;
a first evaluation step C S205 in which each mass spectrum 20 is analyzed for the presence of a cell-specific mass spectrometric signature 41 for the animal cells 23;
a second evaluation step C S206a, in which a degree of coverage 45 by animal cells 23 is determined for at least the partial area 34 of the sample spot 16 from the result of the first evaluation step C S205c;
a third evaluation step A S7, in which a proliferation capability is derived from the determined degree of coverage 45;
a fourth evaluation step C S208, in which the cytotoxic effect 46 of the analytical sample on the animal cells 23 is derived from the determined proliferation capability of the third evaluation step A S7 and thus indirectly from the degree of coverage 45.

FIG. 12c shows a particularly preferred alternative embodiment of the method in which the following steps are carried out: Sample provision step A S1, cultivation step S2, liquid removal step S3a, sample preparation step S4a, measurement step C S204b, first evaluation step C S205, second evaluation step C S206a, fourth evaluation step C S208. The cytotoxic effect 19 is derived directly from the degree of coverage 45 in the fourth evaluation step C S208. This preferred embodiment of the method is used for matrix-based mass spectrometers. Before the measuring step C S204b, a true-to-position application of a matrix 29 takes place in the sample preparation step A S4a.

The process thus has the following features:

A method for determining the cytotoxic effect 46 of an analytical sample on animal cells 23, comprising the following steps:
a sample provision step A S1 in which a mass spectrometric sample 11 comprising animal cells 23, culture medium and the analytical sample potentially having a cytotoxic factor is provided on at least one sample spot 16 of a mass spectrometric sample support 6;
a cultivation step S2 in which the mass spectrometric sample 11 is incubated on the sample spot 16 of the mass spectrometric sample support 6 in a cultivation device 24;
a liquid removal step S3a, in which residual liquid of the mass spectrometric sample 11 is removed from the sample spot 16;
sample preparation step A S4a, in which a preparation of the sample spot 16 is conducted by the true-to-position application of a matrix 29 to at least the one partial area 34 of the sample spot 16;
a measuring step C S204b, in which mass spectra 20 are recorded at a plurality of measuring positions 33 distributed over at least a partial area 34 of the sample spot 16 by means of a matrix-based mass spectrometer;
a first evaluation step C S205 in which each mass spectrum 20 is analyzed for the presence of a cell-specific mass spectrometric signature 41 for the animal cells 23;
a second evaluation step C S206a, in which a degree of coverage 45 by animal cells 23 is determined for at least the partial area 34 of the sample spot 16 from the result of the first evaluation step C S205c;
a third evaluation step A S7, in which a proliferation capability is derived from the determined degree of coverage 45;
a fourth evaluation step C S208, in which the cytotoxic effect 46 of the analytical sample on the animal cells 23 is derived directly from the determined degree of coverage 45 of the second evaluation step C S206a.

FIG. 12d shows a preferred alternative embodiment of the method in which the following steps are performed: Sample provision step A S1, cultivation step S2, liquid removal step S3a, sample preparation step A S4a, measurement step C S204b, first evaluation step C S205, second evaluation step C S206a, third evaluation step A S7, fourth evaluation step C S208. The cytotoxic effect 19 is derived in the fourth evaluation step C S208 from the proliferation capability of the animal cells and thus indirectly from the degree of coverage 45. This preferred embodiment of the method is used for matrix-based mass spectrometers. Before measurement step C S204b, a true-to-position application of a matrix 29 takes place in sample preparation step A S4a.

The process thus has the following features:

A method for determining the cytotoxic effect 46 of an analytical sample on animal cells 23, comprising the following steps:
a sample provision step A S1 in which a mass spectrometric sample 11 comprising animal cells 23, culture medium and the analytical sample potentially having a cytotoxic factor is provided on at least one sample spot 16 of a mass spectrometric sample support 6;
a cultivation step S2 in which the mass spectrometric sample 11 is incubated on the sample spot 16 of the mass spectrometric sample support 6 in a cultivation device 24;
a liquid removal step S3a, in which residual liquid of the mass spectrometric sample 11 is removed from the sample spot 16;
sample preparation step A S4a, in which a preparation of the sample spot 16 is carried out by the true-to-position application of a matrix 29 to at least the one partial area 34 of the sample spot 16;
a measuring step C S204b, in which mass spectra 20 are recorded at a plurality of measuring positions 33 distributed over at least a partial area 34 of the sample spot 16 by means of a matrix-based mass spectrometer;
a first evaluation step C S205 in which each mass spectrum 20 is analyzed for the presence of a cell-specific mass spectrometric signature 41 for the animal cells 23;
a second evaluation step C S206a, in which a degree of coverage 45 by animal cells 23 is determined for at least the partial area 34 of the sample spot 16 from the result of the first evaluation step C S205c;
a third evaluation step A S7, in which a proliferation capability is derived from the determined degree of coverage 45;
a fourth evaluation step C S208, in which the cytotoxic effect 46 of the analytical sample on the animal cells 23 is derived from the determined proliferation capability, of the third evaluation step A S7, thus indirectly from the degree of coverage 45.

Illustrative embodiments of the invention include the following:

According to a first illustrative embodiment, provided is a method for determining the cytotoxic effect (46) of an analytical sample on animal cells (23), comprising the following steps:
a sample provision step A (S1) in which a mass spectrometric sample (11) comprising animal cells (23), culture medium, and the analytical sample potentially having a cytotoxic factor is provided on at least one sample spot (16) of a mass spectrometric sample support (6);
a cultivation step (S2) in which the mass spectrometric sample (11) is incubated on the sample spot (16) of the mass spectrometric sample support (6) in a cultivation device (24);
a liquid removal step (S3a), in which residual liquid of the mass spectrometric sample (11) is removed from the sample spot (16);
a measuring step A (S4b), in which spatially resolved mass spectra (38) are recorded at a plurality of measuring positions (33) in at least a partial area (34) of the sample spot (16) by means of a spatial resolution mass spectrometer (1), wherein, when using a matrix-based spatial resolution mass spectrometer (21), prior to the recording of the spatially resolved mass spectra (21) in the measuring step A (S4b), in a preceding sample preparation step A (S4a) a preparation of the sample spot (16) is carried out by a true-to-position application of a matrix (29) to at least the one partial area (34) of the sample spot (16);
a first evaluation step A (S5) in which each spatially resolved mass spectrum (38) is analyzed for the presence of a cell-specific mass spectrometric signature (41) for the animal cells (23) and a cell presence value (42) is assigned to each measuring position (33);
a second evaluation step A (S6a), in which a degree of coverage (45) by animal cells (23) is determined for at least the partial area (34) of the sample spot (16) from the cell presence values (42) of the measuring positions (33);
and/or a third evaluation step A (S7) in which a proliferation capability is derived from the determined degree of coverage (45);
a fourth evaluation step A (S8), in which the cytotoxic effect (46) of the analytical sample on the animal cells (23) is derived indirectly or directly from the determined degree of coverage (45) of the second evaluation step A (S6a), wherein for this purpose the cytotoxic effect (46) of the analytical sample on the animal cells (23) is derived from the result of at least one of the two steps: second evaluation step A (S6a), third evaluation step A (S7).

According to a second illustrative embodiment, provided is a method for determining the cytotoxic effect (46) of an analytical sample on animal cells (23), comprising the following steps:
a sample provision step A (S1) in which a mass spectrometric sample (11) comprising animal cells (23), culture medium, and the analytical sample potentially having a cytotoxic factor is provided on at least one sample spot (16) of a mass spectrometric sample support (6);
a cultivation step (S2) in which the mass spectrometric sample (11) is incubated on the sample spot (16) of the mass spectrometric sample support (6) in a cultivation device (24);
a liquid removal step (S3a), in which residual liquid of the mass spectrometric sample (11) is removed from the sample spot (16);
a measuring step C (S204b), in which mass spectra (20) are recorded at a plurality of measuring positions (33) distributed over at least a partial area (34) of the sample spot (16) by means of a mass spectrometer, wherein, when a matrix-based mass spectrometer is used, the sample spot (16) is prepared in a preceding sample preparation step A (S4a) by a true-to-position application of a matrix (29) to at least the one partial area (34) of the sample spot (16);
a first evaluation step C (S205) in which each mass spectrum (20) is analyzed for the presence of a cell-specific mass spectrometric signature (41) for the animal cells (23);
a second evaluation step C (S206a), in which a degree of coverage (45) by animal cells (23) is determined for at least the partial area (34) of the sample spot (16) from the result of the first evaluation step C (S205);
a fourth evaluation step C (S208), in which the cytotoxic effect (46) of the analytical sample on the animal cells (23) is derived from the determined degree of coverage (45) of the second evaluation step C (S206a).

According to a third illustrative embodiment, provided is a method for determining the cytotoxic effect (56) of an analytical sample on animal cells (23) comprising the following steps:
a sample provision step B (S101) in which a mass spectrometric sample (11) comprising animal cells (23), nutrient medium, and said analytical sample potentially having a cytotoxic factor is provided on at least one sample spot (16) of a mass spectrometric sample support (6), said sample spot (16) having a first and a second partial area (47, 48), the animal cells (23) being provided on said first partial area (47) of said sample spot (16) and no animal cells (23) being provided on said second partial area (48) of said sample spot (16), and the remaining mass spectrometric sample (11) being provided on both partial areas (47, 48);
a cultivation step (S2) in which the mass spectrometric sample (11) is incubated on the sample spot (16) of the mass spectrometric sample support (6) in a cultivation device (24);
a liquid removal step (S3a) in which residual liquids of the mass spectrometric sample (11) on the sample spot (16) are removed;
a measuring step B (S104b) in which spatially resolved mass spectra (38) are recorded at at least two measuring positions (56, 57) of the sample spot (16) by means of a spatial resolution mass spectrometer (1), the first measuring position (33, 56) still having animal cells (23) and the second measuring position (33, 57) having no animal cells (23),
wherein both measuring positions (56, 57) are located on one direction of extension (50) starting from a reference point (51), wherein, when using a matrix-based spatial resolution mass spectrometer (1), prior to the recording of the spatially resolved mass spectra (38) in the measuring step B (S104b), in a preceding sample preparation step B (S104a) a preparation of the sample spot (16) is carried out by a true-to-position application of a matrix (29) to at least a part of both partial areas (47, 48) of the sample spot (48);
a first evaluation step A (S5) in which each spatially resolved mass spectrum (38) is analyzed for the presence of a cell-specific mass spectrometric signature (41) for the animal cells (23) and a cell presence value (42) is assigned to each measuring position (33, 56, 57);
a second evaluation step B (S106a) in which a cell expansion distance (53) is determined from at least one distance from a reference point (51) to a transition point (52), wherein each transition point (52) is calculated from at least the first and second measuring positions (56, 57);
a fourth evaluation step B (S108), in which the cytotoxic effect (46) of the analytical sample is derived from the result of the preceding evaluation step.

According to a fourth illustrative embodiment, provided is a method according to any one of the first to third illustrative embodiments, characterized in that the animal cells (23) are adherently growing animal cells.

According to a fifth illustrative embodiment, provided is a method of according to the first, second, and fourth embodiments, characterized in that the plurality of measuring positions (33) in the at least one partial area (34) of the sample spot (16) are distributed spatially in such a way that the determined degree of coverage (45) is representative of the partial area (34) and/or the sample spot (16).

According to a sixth illustrative embodiment, provided is a method according to any one of the first to fifth embodiments, characterized in that the mass spectrometric sample (11) in the sample provision step A, B (S1, S101) further comprises a potential cytotoxicity factor-neutralizing factor.

According to a seventh illustrative embodiment, provided is a method according to any one of the first to sixth embodiments, characterized in that in addition to the mass spectrometric sample (11) comprising the analytical sample, at least one reference sample is processed by means of the method.

According to an eighth illustrative embodiment, provided is a method according to the seventh illustrative embodiment, characterized in that the reference sample does not comprise the potentially cytotoxic factor and/or the potentially cytotoxic factor-neutralizing factor and, after the determination of the degree of coverage (45) in the second evaluation step A, C (S6a, S206a), a comparison of the degree of coverage (45) of the reference sample to the degree of coverage (45) of the mass spectrometric sample (11) comprising the analytical sample is carried out in an intermediate evaluation step A (S6b) and the comparison is included in the derivation of the proliferation capability in the third evaluation step A (S7) and/or in the derivation of the cytotoxic effect (46) in the fourth evaluation step A, C (S8, S108).

According to a ninth illustrative embodiment, provided is a method according to any one of the first to eighth illustrative embodiments, characterized in that the mass spectrometric sample (11) is provided in the sample provision step A, B (S1, S101) in that the animal cells (23) are already present on the sample spot (16) before application of the culture medium and the analytical sample; or in that the animal cells (23) are applied in suspended form to the sample spot (16).

According to a tenth illustrative embodiment, provided is a method of any one of the first to ninth illustrative embodiments, characterized in that the animal cells (23) are provided by applying the animal cells (23) in suspended form to the sample spot (16) in the sample provision step A, B (S1, S101) and, in a washing step (S3B), the animal cells (23) are washed on the sample support (16) and residual washing liquids are removed, wherein the washing step (S3B) follows or substitutes the liquid removal step (S3a).

According to an eleventh illustrative embodiment, provided is a method according to any one of the first to tenth embodiments, characterized in that the analytical sample is provided by providing a source sample, components of the source sample, or an isolated cytotoxic factor.

According to a twelfth illustrative embodiment, provided is a method according to any one of the first to eleventh illustrative embodiments, characterized in that the source sample is selected from the following group:
Sample of a human
Sample of an animal
Environmental sample

According to a thirteenth illustrative embodiment, provided is a method according to any one of the first to twelfth embodiments, characterized in that the animal cells (23) are vertebrate cells, mammalian cells, and/or human cells.

According to a fourteenth illustrative embodiment, provided is a method according to any one of the first to thirteenth embodiments, characterized in that the animal cells (23) are continuous cell lines or originate from tissue samples taken or tumor samples taken from a human or animal.

According to a fifteenth embodiment, provided is a method according to any one of the first to fourteenth embodiments, characterized in that the at least one cytotoxic factor is from the following group:
chemical element
low-molecular weight chemical compound
high-molecular weight chemical compound
pharmaceutical agent
toxin
archaea
bacterium
virus
fungus
protozoan
algae
parasite
component of microorganisms
secreted substance of microorganisms
cytotoxic substance of biological origin
cytotoxic substance of microbial origin from a human sample
chemotherapeutic agent
antimicrobial agent
antiviral agent
antifungal agent
antibacterial agent
antiparasitic agent
protein
peptide
antibody
antitumor agent
biocide

According to a sixteenth illustrative embodiment, provided is a method according to any one of the first to fifteenth embodiments, characterized in that the analytical sample comprises bacterial cells or components thereof as a cytotoxic factor and the cytotoxicity factor-neutralizing factor is an antibacterial agent directed against the bacterial cells or components thereof; or
in that the analytical sample comprises viral particles or components thereof as a cytotoxic factor and the cytotoxic factor-neutralizing factor is an antiviral agent directed against the viral particles or components thereof; or
in that the analytical sample comprises fungal cells or components thereof as the cytotoxic factor and the cytotoxic factor-neutralizing factor is an antifungal agent directed against the fungal cells or components thereof; or
in that the analytical sample comprises a parasite as the cytotoxic factor and the cytotoxic factor-neutralizing factor is an antiparasitic agent directed against the parasite.

According to a seventeenth illustrative embodiment, provided is a method according to any one of the first to sixteenth embodiments, characterized in that a concentration-dependent cytotoxic effect (46) of the analytical sample is determined by providing different concentrations of the analytical sample respectively on different sample spots (16), and the concentration-dependent cytotoxic effect (46) is a function of the determined degrees of coverage (45) or of the determined cell expansion distances (53) of the different sample spots (16) and the respective different concentrations of the analytical sample.

According to an eighteenth illustrative embodiment, provided is a method according to any one of the first or third to seventeenth illustrative embodiments, characterized in that the spatial resolution mass spectrometer (1) is selected from the following group:
LDI mass spectrometer
MALDI mass spectrometer
DESI mass spectrometer
MALDESI mass spectrometer
SIMS
SIMS imaging mass spectrometer
MALDI-ToF mass spectrometer (2)
MALDI-ToF-ToF mass spectrometer
MALDI-MSI mass spectrometer
Spatial resolution mass spectrometer (1) having a micro-fluid sample ionization device.

According to a nineteenth illustrative embodiment, provided is a method according to any one of the first to eighteenth illustrative embodiments, characterized in that the true-to-position application of the matrix (29) is conducted by means of a spraying process, sublimation process, or sequential directional positioning of a plurality of matrix microdroplets (30) or matrix nanodroplets on the sample spot (16).

According to a twentieth illustrative embodiment, provided is a method according to the third illustrative embodiment, characterized in that the method comprises a third evaluation step B (S107) and/or an intermediate evaluation step B, wherein in the third evaluation step B (S107) a proliferation capability and/or a migration capability of the animal cells (23) is derived from the determined cell expansion distance (53) of the second evaluation step B (S106a) and the cytotoxic effect (46) of the analytical sample is derived in the fourth evaluation step B (S108) from the determined proliferation capability and/or the migration capability of the animal cells (23) and/or the cell expansion distance (53), and/or wherein in an intermediate evaluation step B (S106b) a comparison of the cell expansion distance (53) of at least one reference sample with the cell expansion distance (53) of the mass spectrometric sample (11) comprising the analytical sample is carried out, wherein the comparison is included in the derivation of the proliferation capability and/or the migration capability in the third evaluation step B (S107) and/or in the derivation of the cytotoxic effect (46) in fourth evaluation step B (S108).

### REFERENCE LIST

1 Spatial resolution mass spectrometer
2 MALDI-ToF mass spectrometer
3 MALDI ion source
4 Flight tube
5 Detector
6 Mass spectrometric sample support
7 XY table
8 Laser
9 Laser beam
10 Mirror/lens system
11 Mass spectrometric sample
12 Sample ions
13 Accelerating electrode
14 Time-of-flight mass analyzer
15 Vacuum pump
16 Sample spot
17 Acceleration path
20 Mass spectrum—not spatially resolved
21 Matrix-based spatial resolution mass spectrometer
22 Application device
23 Animal cells
24 Cultivation device
25 Adherent animal cell
26 Non-adherent animal cell
27 Liquid suction device
28 Spraying device
29 Matrix
30 Matrix microdroplets
31 Disrupted animal cell
33 Measuring position
34 Partial area of the sample spot
35 Grid
37 Data processing unit
38 Spatially resolved mass spectrum
39 Mass spectrometric signal
40 Cell-specific mass spectrometric signal
41 Cell-specific mass spectrometric signature
42 Cell presence value
43 Positive cell presence value
44 Negative cell presence value
45 Degree of coverage
46 Cytotoxic effect
47 First partial area
48 Second partial area
49 Boundary line (illustrative)
50 Direction of extension
51 Reference point
52 Transition point
53 Cell expansion distance
55 Non-cell-specific mass spectrometric signal
56 First measuring position
57 Second measuring position
58 Background noise
101 Mass spectrometer
S1 Sample provision step A
S2 Cultivation step
S3a Liquid removal step
S3b Washing step
S4a Sample preparation step A
S4b Measuring step A
S5 First evaluation step A
S6a Second evaluation step A
S6b Intermediate evaluation step A
S7 Third evaluation step A
S8 Fourth evaluation step A
S101 Sample provision step B
S104a Sample preparation step B
S104b Measuring step B
S106a Second evaluation step B
S106b Intermediate evaluation step B
S107 Third evaluation step B
S108 Fourth evaluation step B
S204b Measuring step C
S205 First evaluation step A
S206a Second evaluation step C
S208 Fourth evaluation step C

1. A method for determining the cytotoxic effect (46) of an analytical sample on animal cells (23), comprising the following steps:
a sample provision step A (S1) in which a mass spectrometric sample (11) comprising animal cells (23), culture medium, and the analytical sample potentially having a cytotoxic factor is provided on at least one sample spot (16) of a mass spectrometric sample support (6);
a cultivation step (S2) in which the mass spectrometric sample (11) is incubated on the sample spot (16) of the mass spectrometric sample support (6) in a cultivation device (24);
a liquid removal step (S3a), in which residual liquid of the mass spectrometric sample (11) is removed from the sample spot (16);
a measuring step A (S4b), in which spatially resolved mass spectra (38) are recorded at a plurality of measuring positions (33) in at least a partial area (34) of the sample spot (16) by means of a spatial resolution mass spectrometer (1), wherein, when using a matrix-based spatial resolution mass spectrometer (21), prior to the recording of the spatially resolved mass spectra (21) in the measuring step A (S4b), in a preceding sample preparation step A (S4a) a preparation of the sample spot (16) is carried out by a true-to-position application of a matrix (29) to at least the one partial area (34) of the sample spot (16);
a first evaluation step A (S5) in which each spatially resolved mass spectrum (38) is analyzed for the presence of a cell-specific mass spectrometric signature (41) for the animal cells (23) and a cell presence value (42) is assigned to each measuring position (33);
a second evaluation step A (S6a), in which a degree of coverage (45) by animal cells (23) is determined for at least the partial area (34) of the sample spot (16) from the cell presence values (42) of the measuring positions (33);
and/or a third evaluation step A (S7) in which a proliferation capability is derived from the determined degree of coverage (45);
a fourth evaluation step A (S8), in which the cytotoxic effect (46) of the analytical sample on the animal cells (23) is derived indirectly or directly from the determined degree of coverage (45) of the second evaluation step A (S6a), wherein for this purpose the cytotoxic effect (46) of the analytical sample on the animal cells (23) is derived from the result of at least one of the two steps: second evaluation step A (S6a), third evaluation step A (S7).

2. A method for determining the cytotoxic effect (46) of an analytical sample on animal cells (23), comprising the following steps:
a sample provision step A (S1) in which a mass spectrometric sample (11) comprising animal cells (23), culture medium, and the analytical sample potentially having a cytotoxic factor is provided on at least one sample spot (16) of a mass spectrometric sample support (6);
a cultivation step (S2) in which the mass spectrometric sample (11) is incubated on the sample spot (16) of the mass spectrometric sample support (6) in a cultivation device (24);
a liquid removal step (S3a), in which residual liquid of the mass spectrometric sample (11) is removed from the sample spot (16);
a measuring step C (S204b), in which mass spectra (20) are recorded at a plurality of measuring positions (33) distributed over at least a partial area (34) of the sample spot (16) by means of a mass spectrometer, wherein, when a matrix-based mass spectrometer is used, the sample spot (16) is prepared in a preceding sample preparation step A (S4a) by a true-to-position application of a matrix (29) to at least the one partial area (34) of the sample spot (16);
a first evaluation step C (S205) in which each mass spectrum (20) is analyzed for the presence of a cell-specific mass spectrometric signature (41) for the animal cells (23);
a second evaluation step C (S206a), in which a degree of coverage (45) by animal cells (23) is determined for at least the partial area (34) of the sample spot (16) from the result of the first evaluation step C (S205);
a fourth evaluation step C (S208), in which the cytotoxic effect (46) of the analytical sample on the animal cells (23) is derived from the determined degree of coverage (45) of the second evaluation step C (S206a).

3. A method for determining the cytotoxic effect (56) of an analytical sample on animal cells (23) comprising the following steps:
a sample provision step B (S101) in which a mass spectrometric sample (11) comprising animal cells (23), nutrient medium, and said analytical sample potentially having a cytotoxic factor is provided on at least one sample spot (16) of a mass spectrometric sample support (6), said sample spot (16) having a first and a second partial area (47, 48), the animal cells (23) being provided on said first partial area (47) of said sample spot (16) and no animal cells (23) being provided on said second partial area (48) of said sample spot (16), and the remaining mass spectrometric sample (11) being provided on both partial areas (47, 48);
a cultivation step (S2) in which the mass spectrometric sample (11) is incubated on the sample spot (16) of the mass spectrometric sample support (6) in a cultivation device (24);
a liquid removal step (S3a) in which residual liquids of the mass spectrometric sample (11) on the sample spot (16) are removed;
a measuring step B (S104b) in which spatially resolved mass spectra (38) are recorded at least two measuring positions (56, 57) of the sample spot (16) by means of a spatial resolution mass spectrometer (1), the first measuring position (33, 56) still having animal cells (23) and the second measuring position (33, 57) having no animal cells (23),
wherein both measuring positions (56, 57) are located on one direction of extension (50) starting from a reference point (51), wherein, when using a matrix-based spatial resolution mass spectrometer (1), prior to the recording of the spatially resolved mass spectra (38) in the measuring step B (S104b), in a preceding sample preparation step B (S104a) a preparation of the sample spot (16) is carried out by a true-to-position application of a matrix (29) to at least a part of both partial areas (47, 48) of the sample spot (48);
a first evaluation step A (S5) in which each spatially resolved mass spectrum (38) is analyzed for the presence of a cell-specific mass spectrometric signature (41) for the animal cells (23) and a cell presence value (42) is assigned to each measuring position (33, 56, 57);
a second evaluation step B (S106a) in which a cell expansion distance (53) is determined from at least one distance from a reference point (51) to a transition point (52), wherein each transition point (52) is calculated from at least the first and second measuring positions (56, 57);
a fourth evaluation step B (S108), in which the cytotoxic effect (46) of the analytical sample is derived from the result of the preceding evaluation step.

4. The method according to claim 1, characterized in that the animal cells (23) are adherently growing animal cells.

5. The method according to claim 1, characterized in that the plurality of measuring positions (33) in the at least one partial area (34) of the sample spot (16) are distributed spatially in such a way that the determined degree of coverage (45) is representative of the partial area (34) and/or the sample spot (16).

6. The method according to claim 1, characterized in that the mass spectrometric sample (11) in the sample provision step A (S1) further comprises a potential cytotoxicity factor-neutralizing factor.

7. The method according to claim 1, characterized in that in addition to the mass spectrometric sample (11) comprising the analytical sample, at least one reference sample is processed by means of the method.

8. The method according to claim 7, characterized in that the reference sample does not comprise the potentially cytotoxic factor and/or the potentially cytotoxic factor-neutralizing factor and, after the determination of the degree of coverage (45) in the second evaluation step A (S6a), a comparison of the degree of coverage (45) of the reference sample to the degree of coverage (45) of the mass spectrometric sample (11) comprising the analytical sample is carried out in an intermediate evaluation step A (S6b) and the comparison is included in the derivation of the proliferation capability in the third evaluation step A (S7) and/or in the derivation of the cytotoxic effect (46) in the fourth evaluation step A (S8).

9. The method according to claim 1, characterized in that the mass spectrometric sample (11) is provided in the sample provision step A (S1) in that the animal cells (23) are already present on the sample spot (16) before application of the culture medium and the analytical sample; or in that the animal cells (23) are applied in suspended form to the sample spot (16).

10. The method according to claim 1, characterized in that the animal cells (23) are provided by applying the animal cells (23) in suspended form to the sample spot (16) in the sample provision step A (S1) and, in a washing step (S3B), the animal cells (23) are washed on the sample support (16) and residual washing liquids are removed, wherein the washing step (S3B) follows or substitutes the liquid removal step (S3a).

11. The method according to claim 1, characterized in that the analytical sample is provided by providing a source sample, components of the source sample, or an isolated cytotoxic factor.

12. The method according to claim 1, characterized in that the source sample is selected from the following group:
Sample of a human
Sample of an animal
Environmental sample

13. The method according to claim 1, characterized in that the animal cells (23) are vertebrate cells, mammalian cells, and/or human cells.

14. The method according to claim 1, characterized in that the animal cells (23) are continuous cell lines or originate from tissue samples taken or tumor samples taken from a human or animal.

15. The method according to claim 1, characterized in that the at least one cytotoxic factor is from the following group:
chemical element
low-molecular weight chemical compound
high-molecular weight chemical compound
pharmaceutical agent
toxin
archaea
bacterium
virus
fungus
protozoan
algae
parasite
component of microorganisms
secreted substance of microorganisms
cytotoxic substance of biological origin
cytotoxic substance of microbial origin from a human sample
chemotherapeutic agent
antimicrobial agent
antiviral agent
antifungal agent
antibacterial agent
antiparasitic agent
protein
peptide
antibody
antitumor agent
biocide.

16. The method according to claim 1, characterized in that the analytical sample comprises bacterial cells or components thereof as a cytotoxic factor and the cytotoxicity factor-neutralizing factor is an antibacterial agent directed against the bacterial cells or components thereof; or
in that the analytical sample comprises viral particles or components thereof as a cytotoxic factor and the cytotoxic factor-neutralizing factor is an antiviral agent directed against the viral particles or components thereof; or
in that the analytical sample comprises fungal cells or components thereof as the cytotoxic factor and the cytotoxic factor-neutralizing factor is an antifungal agent directed against the fungal cells or components thereof; or
in that the analytical sample comprises a parasite as the cytotoxic factor and the cytotoxic factor-neutralizing factor is an antiparasitic agent directed against the parasite.

17. The method according to claim 1, characterized in that a concentration-dependent cytotoxic effect (46) of the analytical sample is determined by providing different concentrations of the analytical sample respectively on different sample spots (16), and the concentration-dependent cytotoxic effect (46) is a function of the determined degrees of coverage (45) or of the determined cell expansion distances (53) of the different sample spots (16) and the respective different concentrations of the analytical sample.

18. The method according to claim 1, characterized in that the spatial resolution mass spectrometer (1) is selected from the following group:
LDI mass spectrometer
MALDI mass spectrometer
DESI mass spectrometer
MALDESI mass spectrometer
SIMS
SIMS imaging mass spectrometer
MALDI-ToF mass spectrometer (2)
MALDI-ToF-ToF mass spectrometer
MALDI-MSI mass spectrometer
Spatial resolution mass spectrometer (1) having a micro-fluid sample ionization device.

19. The method according to claim 1, characterized in that the true-to-position application of the matrix (29) is conducted by means of a spraying process, sublimation process, or sequential directional positioning of a plurality of matrix microdroplets (30) or matrix nanodroplets on the sample spot (16).

20. The method according to claim 3, characterized in that the method comprises a third evaluation step B (S107) and/or an intermediate evaluation step B, wherein in the third evaluation step B (S107) a proliferation capability and/or a migration capability of the animal cells (23) is derived from the determined cell expansion distance (53) of the second evaluation step B (S106a) and the cytotoxic effect (46) of the analytical sample is derived in the fourth evaluation step B (S108) from the determined proliferation capability and/or the migration capability of the animal cells (23) and/or the cell expansion distance (53), and/or wherein in an intermediate evaluation step B (S106b) a comparison of the cell expansion distance (53) of at least one reference sample with the cell expansion distance (53) of the mass spectrometric sample (11) comprising the analytical sample is carried out, wherein the comparison is included in the derivation of the proliferation capability and/or the migration capability in the third evaluation step B (S107) and/or in the derivation of the cytotoxic effect (46) in fourth evaluation step B (S108).

21. A system for determining the cytotoxic effect (46) of an analytical sample on animal cells (23) by means of a spatial resolution mass spectrometer (1) comprising at least one spatial resolution mass spectrometer (1) for generating spatially resolved mass spectra (38), a mass spectrometric sample support (6) having sample spots (16), and a data processing unit (37) for controlling the spatial resolution mass spectrometer (1) and for evaluating the spatially resolved mass spectra (38) generated, characterized in that the data processing unit (37) is configured to analyze each spatially resolved mass spectrum (38) with respect to the presence of at least one cell-specific mass spectrometric signature (41) for the animal cells (23); to determine a degree of coverage (45) by the animal cells (23) for at least a partial area (34) of the sample spots (16) and/or a cell expansion distance (53) of the animal cells (23) in at least one direction of extension (50) of the sample spot (16); and to derive the cytotoxic effect (46) of the analytical sample from the degree of coverage (45) and/or from the cell expansion distance (53).
